(19) United States
(12) Patent Application Publication
(10) Pub. No.: US 2018/0049995 A1
(43) Pub. Date: Feb. 22, 2018

(54) HIF-2-ALPHA INHIBITOR POLYMORPHS

(71) Applicant: PELOTON THERAPEUTICS, INC., Dallas, TX (US)

(72) Inventors: Darryl David DIXON, Somerset, NJ (US); Peter J. STENGEL, Irving, TX (US); Bin WANG, Dallas, TX (US)

(21) Appl. No.: 15/556,248

(22) PCT Filed: Mar. 10, 2016
(86) PCT No.: PCT/US16/21846

§ 371 Date: Sep. 6, 2017

Related U.S. Application Data
(60) Provisional application No. 62/131,726, filed on Mar. 11, 2015.

(51) Int. Cl. A61K 31/085; A61K 31/10; C07C 317/22

(52) U.S. Cl. CPC A61K 31/085; A61K 31/10; C07C 2602/08; C07B 2200/13; C07C 317/22

(57) ABSTRACT

Chemical compounds that modulate HIF-2α activity, their polymorphs, pharmaceutical compositions, and methods of treatment of diseases and conditions associated with HIF-2α, are described herein.

### CROSS REFERENCE TO RELATED APPLICATION

This application claims the benefit of U.S. Provisional Application No. 62/131,726, filed on Mar. 11, 2015, incorporated herein by reference in its entirety.

### BACKGROUND OF THE INVENTION

This invention was in part funded by a grant from Cancer Prevention Research Institute of Texas (Grant number R1009).

Intratumoral hypoxia is a driving force in cancer progression and is closely linked to poor patient prognosis and resistance to chemotherapy and radiation treatment. Progress over the past several decades in mapping the molecular mechanisms that enable cellular adaptation to chronic oxygen deprivation has intensified interest in identifying drugs that effectively block the hypoxic response pathway in tumors. Hypoxia-Inducible Factors (HIF-1α and HIF-2α) are transcription factors that play central roles in this pathway, and thus represent attractive targets for therapeutic intervention. The half-life of HIF-α proteins is tightly regulated by the oxidative status within the cell. Under normoxic conditions, specific proline residues on the HIF proteins are hydroxylated by the oxygen sensitive HIF-specific prolyl-hydroxylases (PHD). The tumor suppressor von Hippel-Lindau (VHL) protein binds to the specific hydroxylated proline residues and recruits E3 ubiquition-ligase complex that targets HIF-α proteins for proteasomal degradation. Because PHDs require oxygen to function, under hypoxic conditions, HIF-α proteins accumulate and enter the nucleus to activate gene expression. Genetic mutations of the VHL gene that result in loss of function lead to constitutively active HIF-α proteins regardless of oxygen levels. Upon activation, these transcription factors stimulate the expression of genes that coordinately regulate anaerobic metabolism, angiogenesis, cell proliferation, cell survival, extracellular matrix remodeling, pH homeostasis, amino acid and nucleotide metabolism, and genomic instability. While many gene products involved in the hypoxic response have been explored individually as therapeutic targets for cancer, broad inhibition of the pathway through direct targeting of HIF-α proteins offers an exciting opportunity to attack tumors on multiple fronts (Keith, et al. Nature Rev. Cancer 12: 9-22, 2012).

Both HIF-1α and HIF-2α form a dimeric complex with HIF-1β (or ARNT: aryl hydrocarbon receptor nuclear translocator) and subsequently bind to hypoxia response elements (HRE) in target genes. Because the level of HIF-1β is unaffected by oxygen levels or VHL, transcriptional activity of the complex is largely driven by the availability of the HIF-α proteins. While HIF-1α and HIF-2α share significant sequence homology, they differ in tissue distribution, sensitivity to hypoxia, timing of activation and target gene specificity (Hu, et al. Mol. Cell Biol. 23: 9361-9374, 2003 and Keith, et al. Nature Rev. Cancer 12: 9-22, 2012). Whereas HIF-1α mRNA is ubiquitously expressed, the expression of HIF-2α mRNA is found primarily in kidney fibroblasts, hepatocytes and intestinal lumen epithelial cells. Consistent with the tight regulation of the HIF-α proteins under normal physiology, neither is detected in normal tissue with the exception of HIF-2α in macrophages (Talks, et al. Am. J. Pathol. 157: 411-421, 2000). However, HIF-2α protein has been detected in various human tumors of the bladder, breast, colon, liver, ovaries, pancreas, prostate and kidney as well as tumor-associated macrophages (Talks, et al. Am. J. Pathol. 157: 411-421, 2000). HIF-1α has been reported to give a transient, acute transcriptional response to hypoxia while HIF-2α provides more prolonged transcriptional activity. Furthermore, HIF-2α has greater transcriptional activity than HIF-1α under moderately hypoxic conditions like those encountered in end capillaries (Holmquist-Mengelbier, et al. Cancer Cell 10: 413-423, 2006). Whereas some hypoxia-regulated genes are controlled by both HIF-1α and HIF-2α, some are only responsive to specific HIF-α proteins. For example, lactate dehydrogenase A (LDHA), phosphoglycerate kinase (PGK) and pyruvate dehydrogenase kinase 1 (PDK1) are uniquely controlled by HIF-1α whereas Oct-4 and erythropoietin (EPO) by HIF-2α. Often the relative contributions of the HIF-α proteins to gene transcription are cell type-, and disease-specific. More importantly, the HIF-α proteins may play contrasting roles in tumorigenesis. For example, the oncogene MYC is a transcription factor that controls cell cycle G1/S transition. MYC is overexpressed in 40% of human cancer. It has been shown that HIF-2α activity increases MYC transcription activity whereas HIF-1α inhibits MYC activity. As a result, in MYC driven tumors, HIF-2α inhibition reduced proliferation whereas HIF-1α inhibition increased growth (Gordan, et al. Cancer Cell 11: 335-347, 2007 and Koshiji et al. EMBO J. 23: 1949-1956, 2004).

Therefore, the identification of effective small molecules to modulate the activity of HIF-2α is desirable. While such compounds are often initially evaluated for their activity when dissolved in solution, solid state characteristics such as polymorphism are also important. Polymorphic forms of a drug substance such as an inhibitor of HIF-2α can have different physical properties, including melting point, apparent solubility, dissolution rate, optical and mechanical properties, vapor pressure, and density. These properties can have a direct effect on the ability to process or manufacture a drug substance and the drug product. Moreover, polymorphism is often a factor under regulatory review of the ‘sameness’ of drug products from various manufacturers. For example, polymorphism has been evaluated in many multi-million dollar and even multi-billion dollar drugs, such as warfarin sodium, famotidine, and ranitidine. Polymorphism can affect the quality, safety, and/or efficacy of a drug product, such as a kinase inhibitor.

### SUMMARY OF THE INVENTION

Thus, there still remains a need for polymorphs of HIF-2α inhibitors. This invention addresses this need and provides related advantages as well.

In one aspect, the disclosure provides a composition comprising predominantly polymorph Form A of a compound of Formula I:

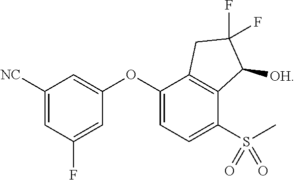 [SMILES: CS(=O)(=O)c1ccc(Oc2cc(F)cc(C#N)c2)c2c1[C@H](O)C(F)(F)C2]

In some embodiments, greater than about 90% of the compound of Formula I is polymorph Form A. In some embodiments, greater than about 95% of the compound of Formula I is polymorph Form A. In some embodiments, greater than about 99% of the compound of Formula I is polymorph Form A. In some embodiments, the polymorph Form A is characterized by having X-ray powder diffraction (XRPD) peaks at about 17.8, about 18.5, about 20.3 and about 21.2 degrees 2θ. In some embodiments, the polymorph Form A is characterized by having X-ray powder diffraction (XRPD) peaks at about 6.8, about 15.9, about 17.8, about 18.5, about 20.3, about 20.5, about 21.2, about 22.1, about 22.7 and about 24.7 degrees 2θ. In some embodiments, the polymorph Form A comprises cubic crystals. In some embodiments, the polymorph Form A has a chemical purity of greater than about 90%. In some embodiments, the polymorph Form A has a chemical purity of greater than about 95%. In some embodiments, the polymorph Form A has a chemical purity of greater than about 99%. In some embodiments, the chemical purity of the polymorph Form A is measured by HPLC analysis. In some embodiments, the polymorph Form A has an enantiomeric purity of greater than about 90%. In some embodiments, the polymorph Form A has an enantiomeric purity of greater than about 95%. In some embodiments, the polymorph Form A has an enantiomeric purity of greater than about 99%. In some embodiments, the polymorph Form A is dry. In some embodiments, the polymorph Form A is non-solvated. In some embodiments, the polymorph Form A is non-hydrated. In some embodiments, the polymorph Form A is non-hygroscopic.

In another aspect, the disclosure provides a composition comprising polymorph Form B of a compound of Formula I:

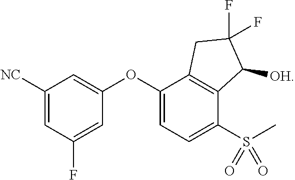 [SMILES: CS(=O)(=O)c1ccc(Oc2cc(F)cc(C#N)c2)c2c1[C@H](O)C(F)(F)C2]

In some embodiments, the polymorph Form B is characterized by having X-ray powder diffraction (XRPD) peaks at about 24.3 degrees 2θ. In some embodiments, the polymorph Form B is characterized by having X-ray powder diffraction (XRPD) peaks at about 12.8 and about 24.3 degrees 2θ. In some embodiments, the polymorph Form B is characterized by having X-ray powder diffraction (XRPD) peaks at about 12.8, about 17.6 and about 24.3 degrees 2θ. In some embodiments, the polymorph Form B is characterized by having X-ray powder diffraction (XRPD) peaks at about 12.8, about 14.8, about 17.6 and about 24.3 degrees 2θ. In some embodiments, the polymorph Form B is characterized by having X-ray powder diffraction (XRPD) peaks at about 12.8, about 14.8, about 17.6, about 20.1, about 20.9, about 22.2, about 24.3, about 25.0, about 25.6 and about 28.1 degrees 2θ. In some embodiments, the polymorph Form B comprises thin rod or needle like crystals. In some embodiments, the polymorph Form B has a chemical purity of greater than about 90%. In some embodiments, the polymorph Form B has a chemical purity of greater than about 95%. In some embodiments, the polymorph Form B has a chemical purity of greater than about 99%. In some embodiments, the chemical purity of the polymorph Form B is measured by HPLC analysis. In some embodiments, the polymorph Form B has an enantiomeric purity of greater than about 90%. In some embodiments, the polymorph Form B has an enantiomeric purity of greater than about 95%. In some embodiments, the polymorph Form B has an enantiomeric purity of greater than about 99%. In some embodiments, the polymorph Form B is dry. In some embodiments, the polymorph Form B is non-solvated. In some embodiments, the polymorph Form B is non-hydrated. In some embodiments, the polymorph Form B is non-hygroscopic. In some embodiments, the composition further comprises polymorph Form A. In some embodiments, the composition further comprises amorphous form of Formula I. In some embodiments, the composition further comprises polymorph Form A and amorphous form of Formula I. In some embodiments, the ratio of polymorph Form B to the total amount of non-B polymorphs is greater than about 1:1. In some embodiments, the ratio of polymorph Form B to the total amount of non-B polymorphs is greater than about 9:1. In some embodiments, the ratio of polymorph Form B to the total amount of non-B polymorphs is greater than about 99:1. In some embodiments, Form B is at least 98% by weight compound of Formula I.

In another aspect, the disclosure provides a composition comprising amorphous polymorph of a compound of Formula I:

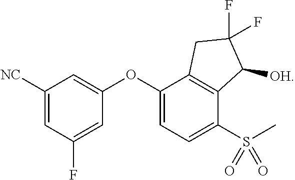 [SMILES: CS(=O)(=O)c1ccc(Oc2cc(F)cc(C#N)c2)c2c1[C@H](O)C(F)(F)C2]

In another aspect, the disclosure provides a pharmaceutical composition comprising a polymorph Form B of a compound of Formula I:

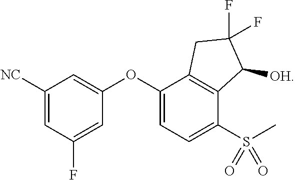 [SMILES: CS(=O)(=O)c1ccc(Oc2cc(F)cc(C#N)c2)c2c1[C@H](O)C(F)(F)C2]

In some embodiments, the composition further comprises one or more non-B polymorphs of the compound of Formula I. In some embodiments, the composition further comprises polymorph Form A. In some embodiments, the composition further comprises amorphous form of Formula I. In some embodiments, the ratio of polymorph Form B to the total amount of non-B polymorphs is greater than about 1:1. In some embodiments, the ratio of polymorph Form B to the total amount of non-B polymorphs is greater than about 9:1. In some embodiments, the composition is in a solid dosage form. In some embodiments, the composition is a suspension. In some embodiments, the composition is an aqueous suspension. In some embodiments, composition is a solution. In some embodiments, the composition, further comprises one or more excipients selected from the group consisting of polysorbate, polyethyleneglycol, cyclodextrin, dextrose, n-methylpyrrolidone, pH buffers, dilute hydrochloric acid, polyoxyethylene esters of 12-hydroxystearic acid, and mixtures thereof. In some embodiments, the composition, further comprises one or more excipients selected from the group consisting of mannitol, microcrystalline cellulose, lactose, dicalcium phosphate, colloidal silicon dioxide, talc, sodium starch glycolate, magnesium stearate, sodium stearyl fumarate, sodium lauryl sulfate, hydroxypropyl methylcellulose, hydroxypropyl cellulose, copovidone, crospovidone, pregelatinized starch, crosscamellose sodium, and polysorbate 80. In some embodiments, the composition further comprises methylcellulose. In some embodiments, the composition further comprises polysorbate 20, polysorbate 21, polysorbate 40, polysorbate 60, polysorbate 61, polysorbate 65, polysorbate 80, polysorbate 81, polysorbate 85 or polysorbate 120. In some embodiments, the composition further comprises polysorbate 80.

In another aspect, the disclosure provides a pharmaceutical composition predominantly comprising Polymorph Form A of a compound of Formula I:

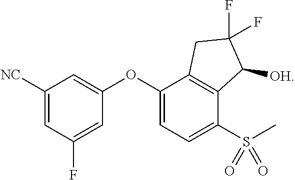 [SMILES: CS(=O)(=O)c1ccc(Oc2cc(F)cc(C#N)c2)c2c1[C@H](O)C(F)(F)C2]

In some embodiments, greater than 90% of the compound of Formula I is polymorph Form A. In some embodiments, greater than 95% of the compound of Formula I is polymorph Form A. In some embodiments, greater than 99% of the compound of Formula I is polymorph Form A. In some embodiments, the composition further comprises one or more non-A polymorphs of the compound of Formula I. In some embodiments, the composition comprises polymorph Form B. In some embodiments, the composition comprises amorphous form of Formula I. In some embodiments, the composition is in a solid dosage form. In some embodiments, the composition is a suspension. In some embodiments, the composition is an aqueous suspension. In some embodiments, the composition is a solution. In some embodiments, the composition further comprises one or more excipients selected from the group consisting of polysorbate, polyethyleneglycol, cyclodextrin, dextrose, n-methylpyrrolidone, pH buffers, dilute hydrochloric acid, polyoxyethylene esters of 12-hydroxystearic acid, and mixtures thereof. In some embodiments, the composition further comprises one or more excipients selected from the group consisting of mannitol, microcrystalline cellulose, lactose, dicalcium phosphate, colloidal silicon dioxide, talc, sodium starch glycolate, magnesium stearate, sodium stearyl fumarate, sodium lauryl sulfate, hydroxypropyl methylcellulose, hydroxypropyl cellulose, copovidone, crospovidone, pregelatinized starch, crosscamellose sodium, and polysorbate 80. In some embodiments, the composition further comprises methylcellulose. In some embodiments, the composition further comprises polysorbate 20, polysorbate 21, polysorbate 40, polysorbate 60, polysorbate 61, polysorbate 65, polysorbate 80, polysorbate 81, polysorbate 85 or polysorbate 120. In some embodiments, the composition further comprises polysorbate 80.

In another aspect, the disclosure provides a method of inhibiting HIF-2α activity in a cell, comprising contacting said cell with an effective amount of a composition or pharmaceutical composition of the disclosure.

In another aspect, the disclosure provides a method of inhibiting HIF-2α, comprising contacting HIF-2α with an effective amount of a compound of a composition or pharmaceutical composition of the disclosure, wherein inhibition of HIF-2α is evidenced by a reduction in the mRNA level of a HIF-2α-regulated gene.

In another aspect, the disclosure provides a method of inhibiting the heterodimerization of HIF-2α and ARNT, comprising contacting HIF-2α with an effective amount of a composition or pharmaceutical composition of the disclosure, thereby reducing the heterodimerization of HIF-2α and ARNT.

In some embodiments, the step of contacting in the methods of inhibiting HIF-2α or the method of inhibiting the heterodimerization of HIF-2α and ARNT disclosed herein comprises contacting a cell that expresses HIF-2α. In some embodiments, the methods of inhibiting HIF-2α or the method of inhibiting the heterodimerization of HIF-2α and ARNT disclosed herein further comprising administering a second therapeutic agent to the cell. In some embodiments, the contacting step takes place in vivo. In some embodiments, the contacting step takes place in vitro.

In another aspect, the disclosure provides a method of treating a condition associated with HIF-2α, comprising administering to a subject in need thereof an effective amount of a composition or pharmaceutical composition of the disclosure.

In another aspect, the disclosure provides a method of treating a neoplastic condition in a subject, comprising administering to said subject a therapeutically effective amount of a composition or pharmaceutical composition of the disclosure.

In another aspect, the disclosure provides a method of treating renal cell carcinoma (RCC) in a subject, comprising administering to said subject a therapeutically effective amount of a composition or pharmaceutical composition of the disclosure.

In some embodiments, the subject in the various methods disclosed herein is a human.

In some embodiments, the renal cell carcinoma in the various methods disclosed herein is clear cell renal cell carcinoma (ccRCC).

In another aspect, the disclosure provides a method of making polymorph Form B of the compound of Formula I:

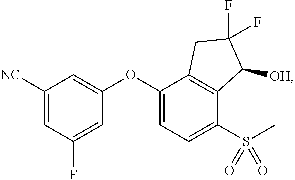 [SMILES: CS(=O)(=O)c1ccc(Oc2cc(F)cc(C#N)c2)c2c1[C@H](O)C(F)(F)C2]

the method comprising: (i) dissolving a composition comprising the compound of Formula I in a solvent to obtain a solution of the compound of Formula I; and (ii) isolating said polymorph Form B from the solution of the compound of Formula I; wherein the polymorph Form B is characterized by having X-ray powder diffraction (XRPD) peaks at about 24.3 degrees 2θ. In some embodiments, the polymorph Form B is characterized by having X-ray powder diffraction (XRPD) peaks at about 12.8, about 14.8, about 17.6, about 20.1, about 20.9, about 22.2, about 24.3, about 25.0, about 25.6 and about 28.1 degrees 2θ. In some embodiments, the solvent comprises a polar protic solvent. In some embodiments, the solvent comprises 2-propanol. In some embodiments, the step of dissolving comprises heating a mixture of the composition comprising the compound of Formula I and the solvent to a temperature above the ambient temperature. In some embodiments, the step of dissolving comprises heating a mixture of the composition comprising the compound of Formula I and the solvent to a temperature of about 70 to about 85° C. In some embodiments, the method further comprises introducing a seed polymorph Form B into the solution of the compound of Formula I, thereby making the polymorph Form B. In some embodiments, the seed polymorph Form B is introduced at a temperature of about 70 to about 72° C. In some embodiments, the method further comprises heating the solution comprising the seed polymorph Form B at a temperature of about 70° C. for about 1 to about 2 hours. In some embodiments, the method further comprises stirring the solution comprising the seed polymorph Form B at a temperature of about 20° C. for about 5-7 hours. In some embodiments, the method further comprises further comprising stirring the solution comprising the seed polymorph Form B at a temperature of about 5-10° C. for about 5-7 hours.

In another aspect, the disclosure provides a method of making polymorph Form A of the compound of Formula I:

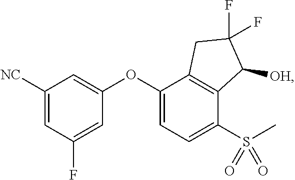 [SMILES: CS(=O)(=O)c1ccc(Oc2cc(F)cc(C#N)c2)c2c1[C@H](O)C(F)(F)C2]

said method comprising: (i) dissolving a composition comprising the compound of Formula I in a solvent to obtain a solution of the compound of Formula I; and (ii) isolating said polymorph Form A, from the solution of the compound of Formula I; wherein said polymorph Form A is characterized by having X-ray powder diffraction (XRPD) peaks at about 17.8, about 18.5, 2 about 0.3 and about 21.2 degrees 2θ. In some embodiments, the polymorph Form A is characterized by having X-ray powder diffraction (XRPD) peaks at about 6.8, about 15.9, about 17.8, about 18.5, about 20.3, about 20.5, about 21.2, about 22.1, about 22.7 and about 24.7 degrees 2θ. In some embodiments, the solvent comprises a polar protic solvent. In some embodiments, the solvent comprises 2-propanol. In some embodiments, the step of dissolving comprises heating a mixture of the composition comprising the compound of Formula I and the solvent to a temperature above the ambient temperature. In some embodiments, the step of dissolving comprises heating a mixture of the composition comprising the compound of Formula I and the solvent to a temperature of about 70 to about 85° C. In some embodiments, the method, further comprises introducing a seed polymorph Form A into the solution of the compound of Formula I, thereby making the polymorph Form A. In some embodiments, the method, further comprises stirring solution comprising the seed polymorph Form A at a temperature of about 5-10° C. for about 5-7 hours.

### DESCRIPTION OF THE DRAWINGS

The novel features of the invention are set forth with particularity in the appended claims. An understanding of the features and advantages of the present invention may be obtained by reference to the following detailed description that sets forth illustrative embodiments, in which the principles of the invention are utilized, and the accompanying drawings of which:

FIG. 1 shows an exemplary synthetic route to a compound of Formula I.

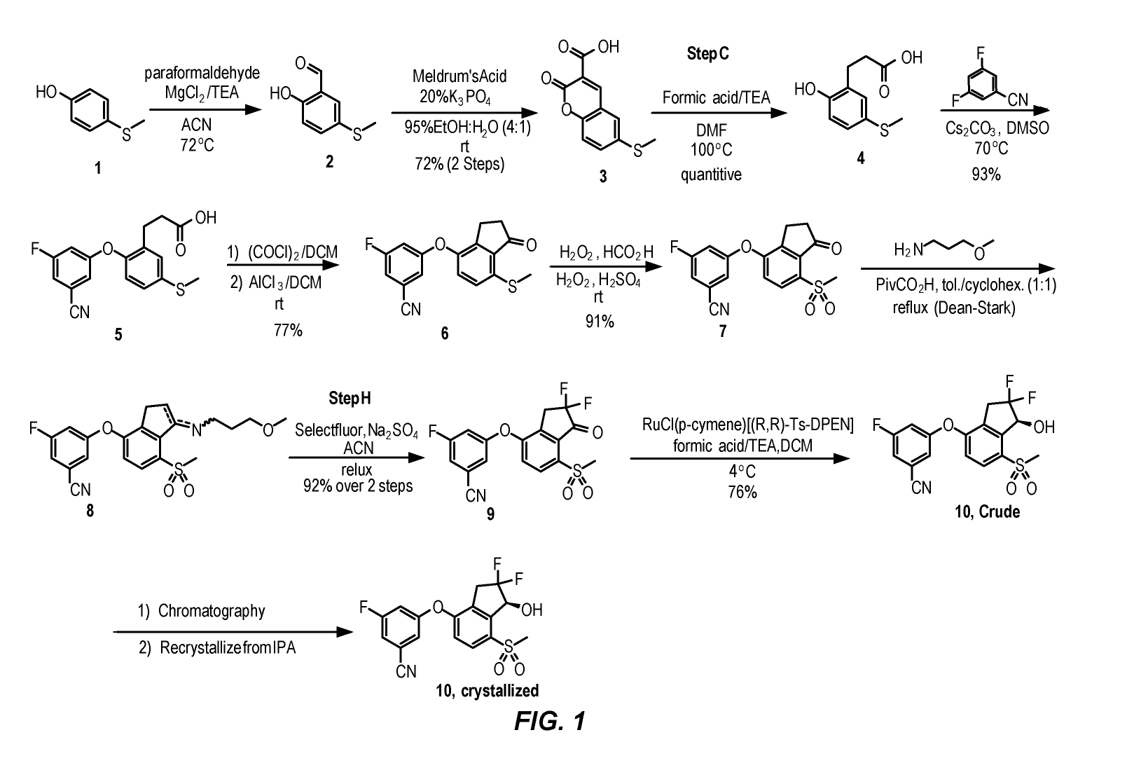

FIG. 2 shows the X-ray powder diffraction (XRPD) for Polymorph Form A of the compound of Formula I.

FIG. 3 shows PLM images of Form A. Non-polarized (left) and between crossed polars (right), 20× magnification (top) and 50× magnification (bottom).

FIG. 4 shows an exemplary HPLC chromatogram of the polymorph Form A of the compound of Formula I.

FIG. 5 shows an exemplary ¹H NMR spectrum of the polymorph Form A of the compound of Formula I.

FIG. 6 shows an exemplary IR spectrum of the polymorph Form A of the compound of Formula I.

FIG. 7 shows an exemplary TG/DTA thermogram of Form A of the compound of Formula I.

FIG. 8 shows an exemplary DSC thermogram of the polymorph Form A of the compound of Formula I.

FIG. 9 shows an exemplary DVS isotherm plot of the polymorph Form A of the compound of Formula I.

FIG. 10 shows a comparison of XRPD diffractogram of Form A of the compound of Formula I pre- and post-DVS analysis.

FIG. 11 shows an exemplary HPLC chromatogram of the polymorph Form B of the compound of Formula I.

FIG. 12 shows PLM analysis of the polymorph Form B of the compound of Formula I.

FIG. 13 shows the XRPD for the Polymorph Form B of the compound of Formula I.

FIG. 14 shows an exemplary TG/DTA thermogram of Form B of the compound of Formula I.

FIG. 15 shows an exemplary DSC thermogram of the polymorph Form B of the compound of Formula I.

FIG. 16 shows a scanning electron microscope micrograph of a mixture of Form A and Form B of the compound of Formula I.

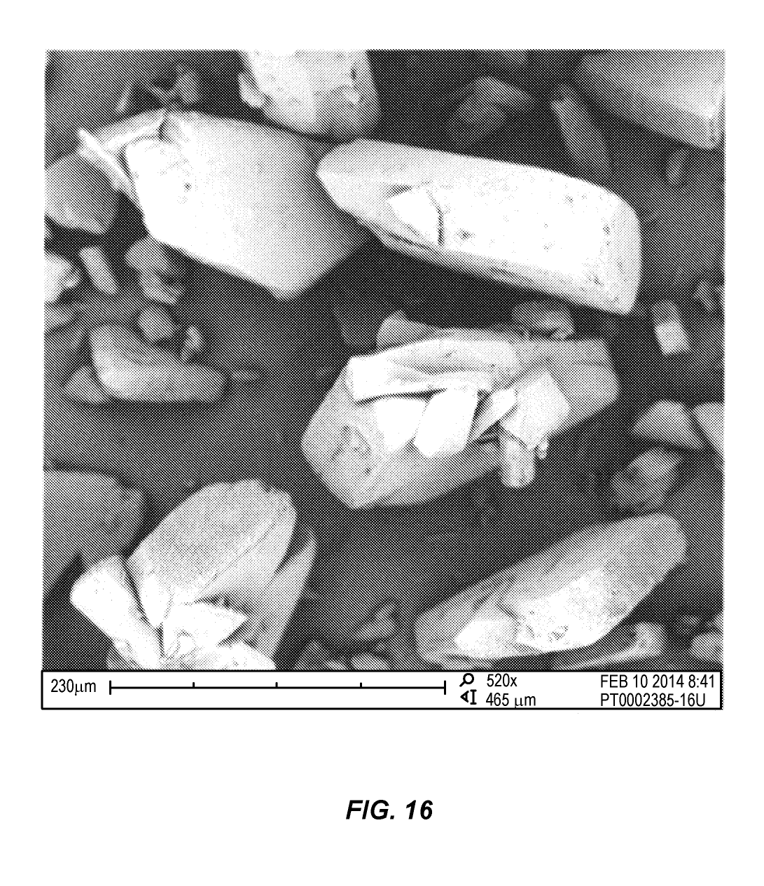

FIG. 17 shows an exemplary IR spectrum of the polymorph Form B of the compound of Formula I.

FIG. 18 shows an exemplary DVS isotherm plot of the polymorph Form B of the compound of Formula I.

FIG. 19 shows a comparison of the properties of Form A and Form B of the compound of Formula I.

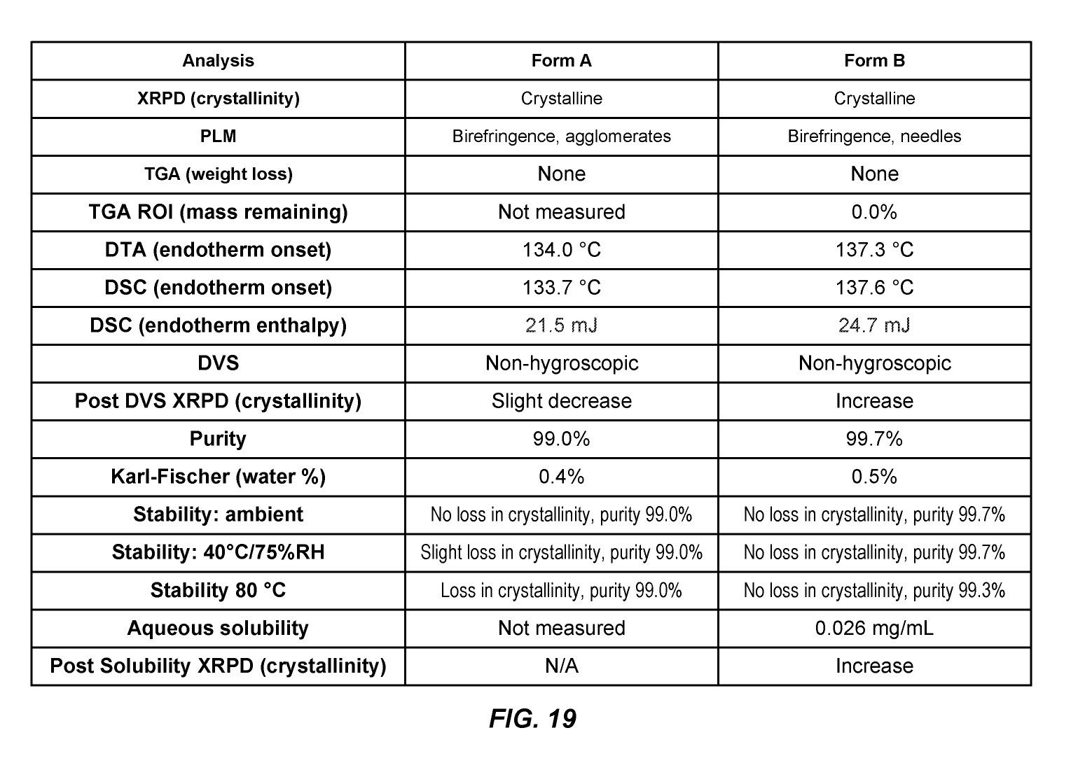

### DETAILED DESCRIPTION OF THE INVENTION

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

### I. Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor, or an undesired immune response as manifested in autoimmune disease.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, the terms “treatment”, “treating”, “palliating” and “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Bis salts (i.e. two counterions) and higher salts are encompassed within the meaning of pharmaceutically acceptable salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “selective inhibition” or “selectively inhibit” as applied to a biologically active agent refers to the agent's ability to selectively reduce the target signaling activity as compared to off-target signaling activity, via direct or interact interaction with the target.

“Subject” refers to an animal, such as a mammal, for example a human. The methods described herein can be useful in both human therapeutics and veterinary applications. In some embodiments, the patient is a mammal, and in some embodiments, the patient is human.

“Radiation therapy” means exposing a patient, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP, or high-energy radiation, including without limitation x-rays, gamma rays, and neutrons.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The term “isolating” also encompasses purifying.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features. The phrase “consists essentially of” excludes unnamed components which materially change the material or composition in major proportions and/or in trace amounts.

As used herein, “predominantly” refers to more than about 50%. In one embodiment, predominantly refers to at least 55%, 60%, 65%, 70%, 75%, 80%, 85%, 90%, 95% or 99%.

The terms “solvent,” “organic solvent,” or “inert solvent” each mean a solvent inert under the conditions of the reaction being described in conjunction therewith including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like. Unless specified to the contrary, the solvents used in the reactions described herein are inert organic solvents. Unless specified to the contrary, for each gram of a limiting reagent, one cc (or mL) of solvent constitutes a volume equivalent.

“Solvate” refers to a compound (e.g., a compound as described herein or a pharmaceutically acceptable salt thereof) in physical association with one or more molecules of a pharmaceutically acceptable solvent.

“Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to. Compounds of the present invention include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof.

Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

In addition, if the compound of the invention is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

When a composition exists as a mixture of polymorphs, the percentage of each polymorphic component may be determined by one or more techniques well known in the art, including, but not limited to, solid state NMR, IR and XRPD.

### II. Compounds and Methods of Making

The chemical entities described herein can generally be synthesized by an appropriate combination of generally well known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Aldrich Chemical Company (Milwaukee, Wis.) or can be readily prepared by those skilled in the art using commonly employed synthetic methodology.

The polymorphs made according to the methods of the invention may be characterized by any methodology according to the art. For example, the polymorphs made according to the methods of the invention may be characterized by X-ray powder diffraction (XRPD), differential scanning calorimetry (DSC), thermogravimetric analysis (TGA), hot-stage microscopy, and spectroscopy (e.g., Raman, solid state nuclear magnetic resonance (ssNMR), and infrared (IR)).

XRPD

Polymorphs according to the invention may be characterized by X-ray powder diffraction patterns (XRPD). The relative intensities of XRPD peaks can vary, depending upon the particle size, the sample preparation technique, the sample mounting procedure and the particular instrument employed. Moreover, instrument variation and other factors can affect the 2−θ values. Therefore, the XRPD peak assignments can vary by plus or minus about 0.2 degrees.

DSC

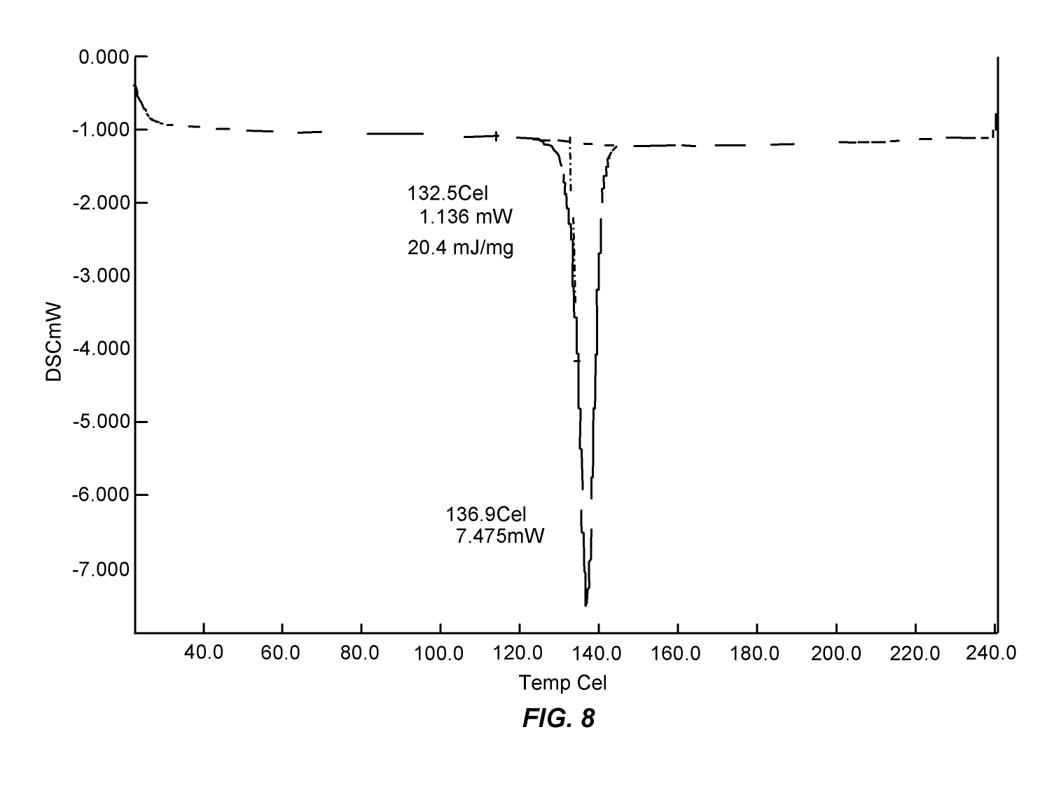

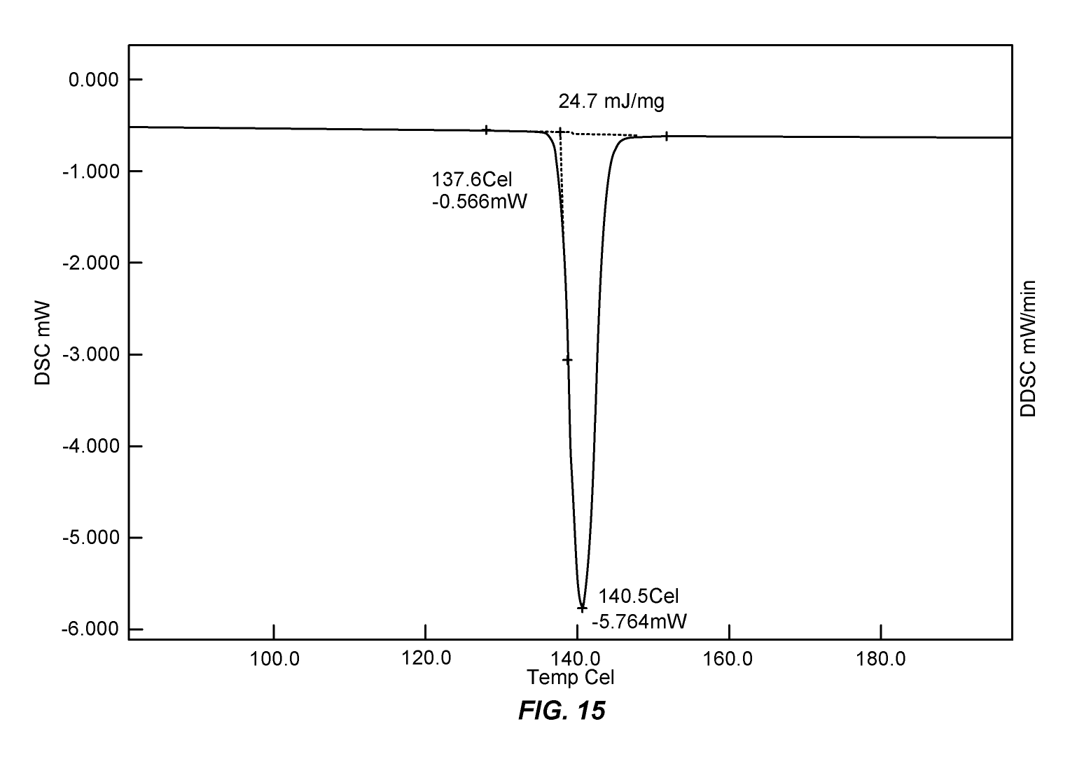

Polymorphs according to the invention can also be identified by its characteristic differential scanning calorimeter (DSC) trace such as shown in FIGS. 8 and 15. For DSC, it is known that the temperatures observed will depend upon the rate of temperature change as well as sample preparation technique and the particular instrument employed. Thus, the values reported herein relating to DSC thermograms can vary by plus or minus about 4° C.

TGA

The polymorphic forms of the invention may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of the invention are useful in the production of medicinal preparations and can be obtained by means of a crystallization process to produce crystalline and semi-crystalline forms or a solidification process to obtain the amorphous form. In various embodiments, the crystallization is carried out by either generating the compound of Formula I in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. The crystallization or solidification may be followed by drying carried out under controlled conditions until the desired water content is reached in the end polymorphic form.

In one aspect, the invention provides methods of making one or more polymorphs of the compound of the Formula I:

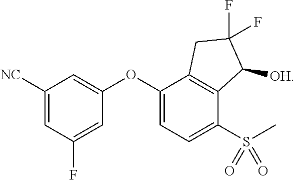 [SMILES: CS(=O)(=O)c1ccc(Oc2cc(F)cc(C#N)c2)c2c1[C@H](O)C(F)(F)C2]

Polymorphs according to the methods of the invention can be selected from Form A, Form B, the amorphous form, and mixtures of more than one form.

In various embodiments, the intermediates for the synthesis of Formula I are made according to the following schemes.

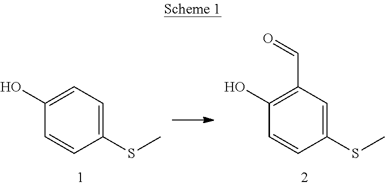

The conversion of compound 1 to compound 2 may be performed according to any method in the art. In one embodiment, compound 1 is treated with paraformaldehyde in the presence of MgCl₂ and triethylamine. In various embodiments, solvent may be acetonitrile.

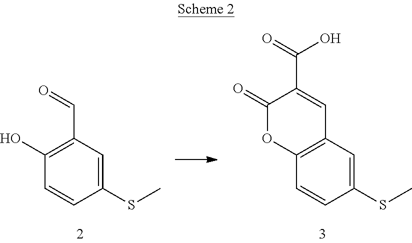 [SMILES: CSc1ccc(O)c(C=O)c1 ; CSc1ccc2oc(=O)c(C(=O)O)cc2c1]

The conversion of compound 2 to compound 3 may be performed according to any method in the art. In one embodiment, the reaction occurs in the presence of 2,2-dimethyl-1,3-dioxane-4,6-dione in presence of tripotassium phosphate.

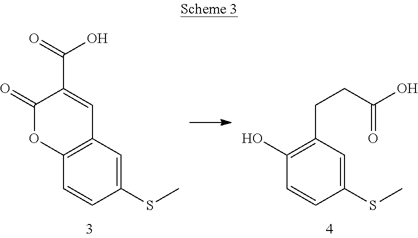 [SMILES: CSc1ccc2oc(=O)c(C(=O)O)cc2c1 ; CSc1ccc(O)c(CCC(=O)O)c1]

The conversion of compound 3 to compound 4 may be performed according to any method in the art. In one embodiment, compound 3 is treated with formic acid in presence of triethylamine to obtain compound 4.

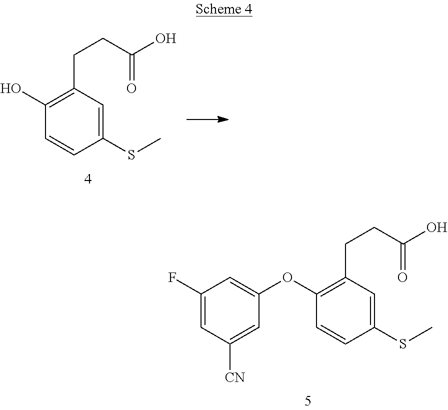 [SMILES: CSc1ccc(O)c(CCC(=O)O)c1 ; CSc1ccc(Oc2cc(F)cc(C#N)c2)c(CCC(=O)O)c1]

The conversion of compound 4 to compound 5 may be performed according to any method in the art. In one embodiment, compound 4 is treated with 3,5-difluorobenzonitrile in presence of caesium carbonate to obtain compound 5.

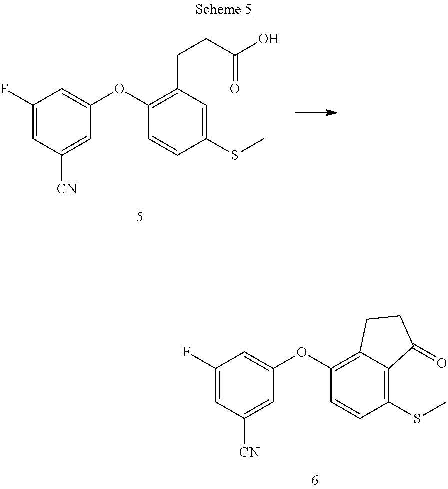 [SMILES: CSc1ccc(Oc2cc(F)cc(C#N)c2)c(CCC(=O)O)c1 ; CSc1ccc(Oc2cc(F)cc(C#N)c2)c2c1C(=O)CC2]

The conversion of compound 5 to compound 6 may be performed according to any method in the art. In one embodiment, compound 5 is treated with oxalyl chloride and with aluminum chloride to obtain compound 6.

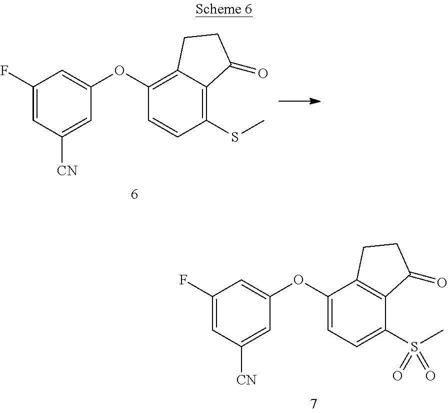 [SMILES: CSc1ccc(Oc2cc(F)cc(C#N)c2)c2c1C(=O)CC2 ; CS(=O)(=O)c1ccc(Oc2cc(F)cc(C#N)c2)c2c1C(=O)CC2]

The conversion of compound 6 to compound 7 may be performed according to any method in the art. In one embodiment, compound 6 is treated with hydrogen peroxide to obtain compound 7.

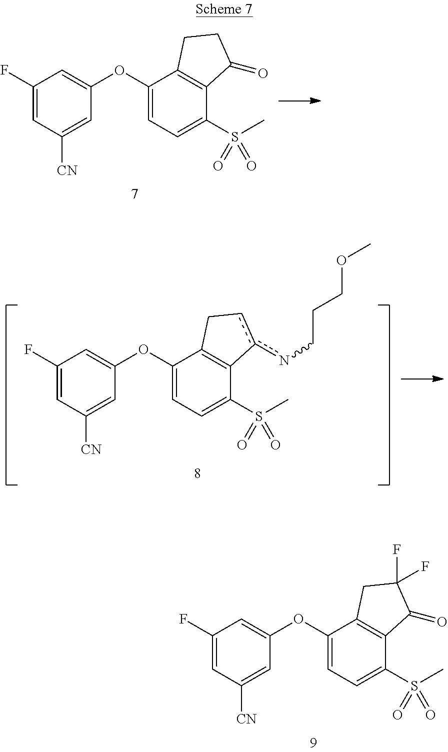

The conversion of compound 7 to compound 9 may be performed according to any method in the art. In one embodiment, compound 7 is treated with 3-methoxypropan-1-amine in presence of PivCOOH to obtain compound 8 which was then treated with Selectfluor® to obtain compound 9.

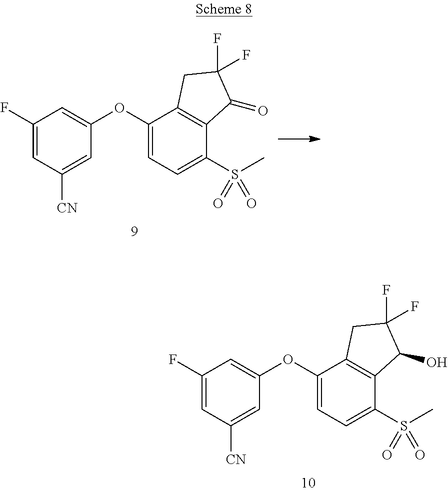 [SMILES: CS(=O)(=O)c1ccc(Oc2cc(F)cc(C#N)c2)c2c1C(=O)C(F)(F)C2 ; CS(=O)(=O)c1ccc(Oc2cc(F)cc(C#N)c2)c2c1[C@H](O)C(F)(F)C2]

The conversion of compound 9 to compound 10 may be performed according to any method in the art. In one embodiment, compound 9 is treated with RuCl(p-cymene)[(R,R)-Ts-DPEN] and Formic acid presence of triethylamine to obtain compound 10.

The polymorphs according to the invention are not limited by the starting materials used to produce the compound of Formula I.

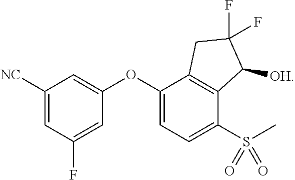 [SMILES: CS(=O)(=O)c1ccc(Oc2cc(F)cc(C#N)c2)c2c1[C@H](O)C(F)(F)C2]

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples below. However, other equivalent separation or isolation procedures can also be used. Prior to formulation as the active pharmaceutical ingredient in a drug product, the compound of Formula I may be isolated in greater than 90% purity, greater than 91% purity, greater than 92% purity, greater than 93% purity, greater than 94% purity, greater than 95% purity, greater than 96% purity, greater than 97% purity, greater than 98% purity, greater than 99% purity, and purity approaching 100%.

When desired, the (R)- and (S)-isomers of the compound of Formula I, if both present, may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation. In certain embodiments, the compound of Formula I is present as a racemic or non-racemic mixture with its enantiomer. In one embodiment, the compound of Formula I is present in enantiomeric excess (EE) selected from greater than 60%, greater than 65%, greater than 70%, greater than 75%, greater than 80%, greater than 85%, greater than 90%, greater than 91%, greater than 92%, greater than 93%, greater than 94%, greater than 95%, greater than 96%, greater than 97%, greater than 98%, greater than 99%, greater than 99.5% and greater than 99.9%.

In one aspect, the invention is directed to methods of making polymorphs of the compound of the Formula I:

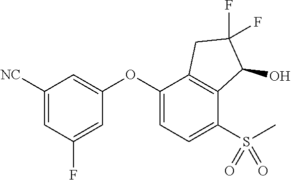 [SMILES: CS(=O)(=O)c1ccc(Oc2cc(F)cc(C#N)c2)c2c1[C@H](O)C(F)(F)C2]

or a pharmaceutically acceptable salt and/or solvate thereof either by isolation of the desired polymorph as the first solid form after synthesis of the compound of Formula I, or alternatively, by isolation of the desired polymorph as a transition from a prior solid form of the compound of Formula I. Transitions from one form to another are within the scope of the invention because they can be an alternative manufacturing method for obtaining the form desired for the production of the medicinal preparations.

Thus, in various embodiments, the methods of the invention include a method of making a polymorph of Formula I comprising reacting a compound of Formula 9:

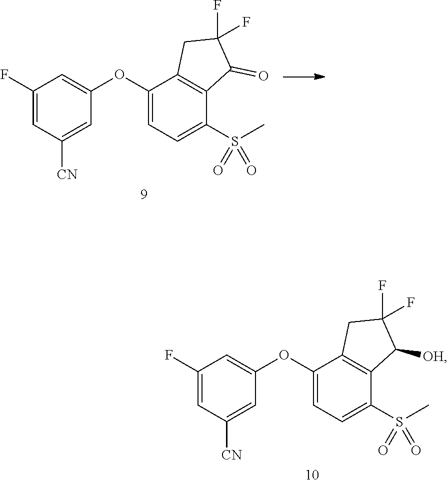 [SMILES: CS(=O)(=O)c1ccc(Oc2cc(F)cc(C#N)c2)c2c1C(=O)C(F)(F)C2 ; CS(=O)(=O)c1ccc(Oc2cc(F)cc(C#N)c2)c2c1[C@H](O)C(F)(F)C2]

with a reagent to reduce the ketone group to yield a compound of Formula I; and isolating the compound of Formula I as the desired polymorph. In various embodiments, the reducing agent may reduce the ketone group asymmetrically.

In one embodiment, the desired polymorph is Form A, and the isolating step involves recrystallization of crude reaction product from a mono-solvent system. In various embodiments, the desired polymorph is Form A, and the isolating step involves recrystallization of crude product from a binary, tertiary, or greater solvent system, collectively understood as a multi-solvent system. In various embodiments, the desired polymorph is Form A, and the isolating step involves crystallization from a mono- or multi-solvent system, where the crystallization involves dissolving the compound of Formula I in the mono- or multi-solvent system at a temperature above ambient temperature. In some examples, the dissolving of the compound of Formula I in the mono- or multi-solvent system is performed at a temperature of about 40-90° C., 50-90° C., 60-90° C., 70-90° C., 80-90° C., 40-80° C., 50-80° C., 60-80° C., 70-80° C., 40-70° C., 50-70° C., 60-70° C., 40-60° C., 50-60° C., or 40-50° C. In some examples, the recrystallization solvent is 2-propanol and the dissolving of the compound of Formula I in the mono- or multi-solvent system is performed at a temperature of about 70-85° C. In various embodiments, the crystallization further involves actively cooling the solution containing the dissolved compound of Formula I, for example to a temperature of about 0-30° C., 5-30° C., 10-30° C., 15-30° C., 20-30° C., 25-30° C., 0-20° C., 5-20° C., 10-20, 15-20° C., 18-20° C., 0-10° C., 5-10° C., or 0-5° C. In various embodiments, the solution containing the dissolved compound of Formula I is further maintained at ambient or lower temperature for some period time, for example for about 30 min, about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h or more. In various embodiments, the desired polymorph is Form A, and the isolating step involves crystallization from a mono- or multi-solvent system, where the crystallization involves addition of an antisolvent either with or without an active cooling step to cause solid Form A to come out of solution. In some examples, the recrystallization solvent is 2-propanol. In various embodiments, the recrystallization also involves addition of Form A seeds to the solution containing the dissolved compound of Formula I. The seeds may be added at a temperature of about 40-90° C., 50-90° C., 60-90° C., 70-90° C., 80-90° C., 40-80° C., 50-80° C., 60-80° C., 70-80° C., 70-72° C., 70-74° C., 70-76° C., 70-78° C., 72-74° C., 72-76° C., 72-78° C., 72-80° C., 74-76° C., 74-78° C., 74-80° C., 76-78° C., 76-80° C., 78-80° C., 40-70° C., 50-70° C., 60-70° C., 40-60° C., 50-60° C., or 40-50° C. In some examples, the recrystallization solvent is 2-propanol

In various embodiments, the desired polymorph is Form B, and the isolating step involves recrystallization of crude reaction product from a mono-solvent system. In various embodiments, the desired polymorph is Form B, and the isolating step involves recrystallization of crude product from a binary, tertiary, or greater solvent system, where binary, tertiary, or greater solvent systems are collectively understood as multi-solvent systems. In various embodiments, the desired polymorph is Form B, and the isolating step involves crystallization from a mono- or multi-solvent system, where the crystallization involves dissolving the compound of Formula I in the mono- or multi-solvent system at a temperature above ambient temperature. In some examples, the dissolving of the compound of Formula I in the mono- or multi-solvent system is performed at a temperature of about 40-90° C., 50-90° C., 60-90° C., 70-90° C., 80-90° C., 40-80° C., 50-80° C., 60-80° C., 70-80° C., 40-70° C., 50-70° C., 60-70° C., 40-60° C., 50-60° C., or 40-50° C. In some examples, the recrystallization solvent is 2-propanol and the dissolving of the compound of Formula I in the mono- or multi-solvent system is performed at a temperature of about 70-85° C. In various embodiments, the crystallization further involves actively cooling the solution containing the dissolved compound of Formula I, for example to a temperature of about 0-30° C., 5-30° C., 10-30° C., 15-30° C., 20-30° C., 25-30° C., 0-20° C., 5-20° C., 10-20° C., 15-20° C., 18-20° C., 0-10° C., 5-10° C., or 0-5° C. In various embodiments, the solution containing the dissolved compound of Formula I is further maintained at ambient or lower temperature for some period time, for example for about 30 min, about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h or more. In various embodiments, the desired polymorph is Form B, and the isolating step involves crystallization from a mono- or multi-solvent system, where the crystallization involves addition of an antisolvent either with or without an active cooling step to cause solid Form B to come out of solution. In some examples, the recrystallization solvent is 2-propanol. In various embodiments, the recrystallization also involves addition of Form B seeds to the solution containing the dissolved compound of Formula I. The seeds may be added at temperature of about 40-90° C., 50-90° C., 60-90° C., 70-90° C., 80-90° C., 40-80° C., 50-80° C., 60-80° C., 70-80° C., 70-72° C., 70-74° C., 70-76° C., 70-78° C., 72-74° C., 72-76° C., 72-78° C., 72-80° C., 74-76° C., 74-78° C., 74-80° C., 76-78° C., 76-80° C., 78-80° C., 40-70° C., 50-70° C., 60-70° C., 40-60° C., 50-60° C., or 40-50° C. In some examples, the recrystallization solvent is 2-propanol.

In various embodiments, the invention is directed to methods of making a polymorph of the compound of Formula I, wherein the method involves converting an isolated polymorph or mixture of polymorphs into a desired polymorph. In certain embodiments, the methods comprise exposing a composition comprising one or more polymorphs to conditions sufficient to convert at least about 50%, 60%, 70%, 80%, 905, 95%, or 99% of the total amount of original polymorph(s) into at least about 50% of the desired polymorph, and isolating the desired polymorph as needed.

In various embodiments, the original solid form of the compound of Formula I contains greater than about 5% polymorph Form A. In some examples, the original solid form of the compound of Formula I contains greater than about 10% polymorph Form A. In some examples, the original solid form of the compound of Formula I contains greater than about 20% polymorph Form A. In some examples, the original solid form of the compound of Formula I contains greater than about 30% polymorph Form A. In some examples, the original solid form of the compound of Formula I contains greater than about 40% polymorph Form A. In some examples, the original solid form of the compound of Formula I contains greater than about 50% polymorph Form A. In some examples, the original solid form of the compound of Formula I contains greater than about 60% polymorph Form A. In some examples, the original solid form of the compound of Formula I contains greater than about 70% polymorph Form A. In some examples, the original solid form of the compound of Formula I contains greater than about 80% polymorph Form A. In some examples, the original solid form of the compound of Formula I contains greater than about 90% polymorph Form A. In some examples, the original solid form of the compound of Formula I contains greater than about 95% polymorph Form A. In some examples, the original solid form of the compound of Formula I contains greater than about 99% polymorph Form A. In some examples, the original solid form of the compound of Formula I is predominantly Form A.

In various embodiments, the original solid form of the compound of Formula I contains greater than about 90% non-Form B polymorphs, and the desired polymorph is Form B. In various embodiments, the original solid form of the compound of Formula I contains greater than about 80% non-Form B polymorphs, and the desired polymorph is Form B. In various embodiments, the original solid form of the compound of Formula I contains greater than about 70% non-Form B polymorphs, and the desired polymorph is Form B. In various embodiments, the original solid form of the compound of Formula I contains greater than about 60% non-Form B polymorphs, and the desired polymorph is Form B. In various embodiments, the original solid form of the compound of Formula I contains greater than about 50% non-Form B polymorphs, and the desired polymorph is Form B. In various embodiments, the original solid form of the compound of Formula I contains greater than about 40% non-Form B polymorphs, and the desired polymorph is Form B. In various embodiments, the original solid form of the compound of Formula I contains greater than about 30% non-Form B polymorphs, and the desired polymorph is Form B. In various embodiments, the original solid form of the compound of Formula I contains greater than about 20% non-Form B polymorphs, and the desired polymorph is Form B. In various embodiments, the original solid form of the compound of Formula I contains greater than about 10% non-Form B polymorphs, and the desired polymorph is Form B.

In various embodiments, the invention is directed to compositions comprising a mixture of more than one polymorph of the compound of Formula I. For example, in various embodiments, the composition comprises a ratio of Form B to non-B polymorphs where the ratio is greater than 1:1, or greater than 9:1, or greater than 99:1. In various embodiments, the composition comprises both Form B and Form A.

### Form A

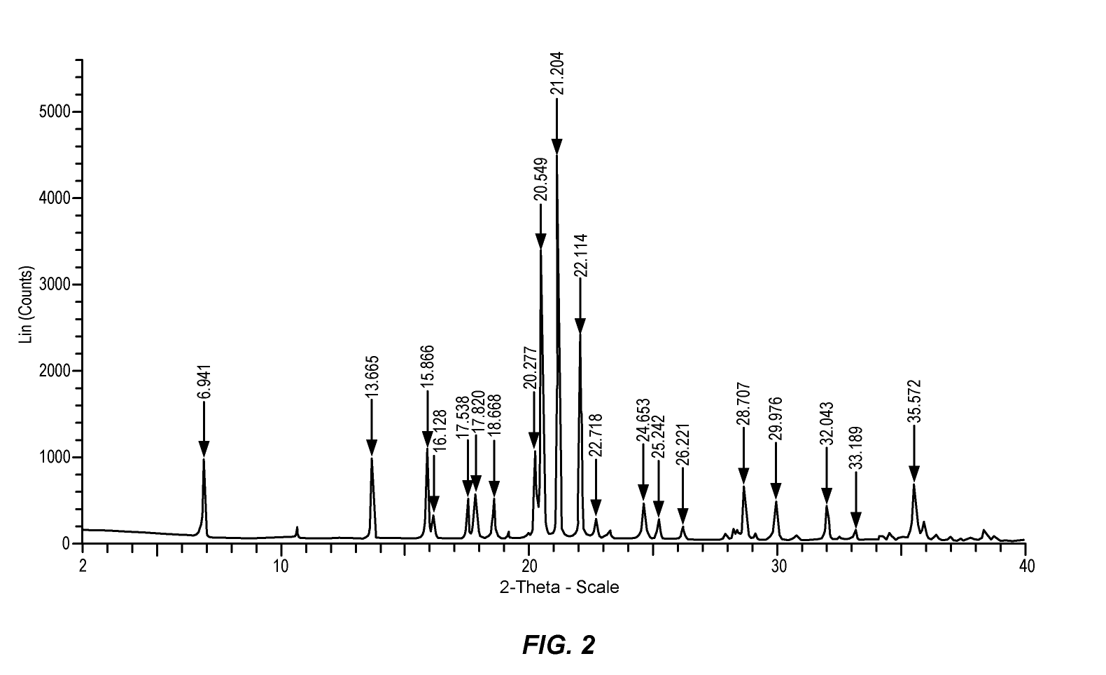

FIG. 2 shows the X-ray powder diffraction (XRPD) for Polymorph Form A.

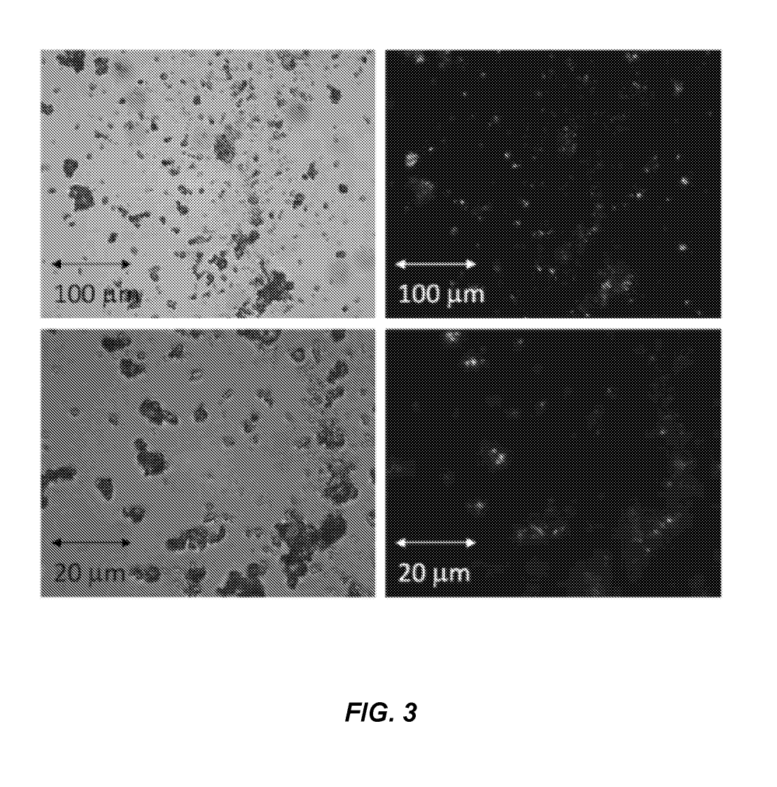

FIG. 3 shows exemplary PLM Images of Form A.

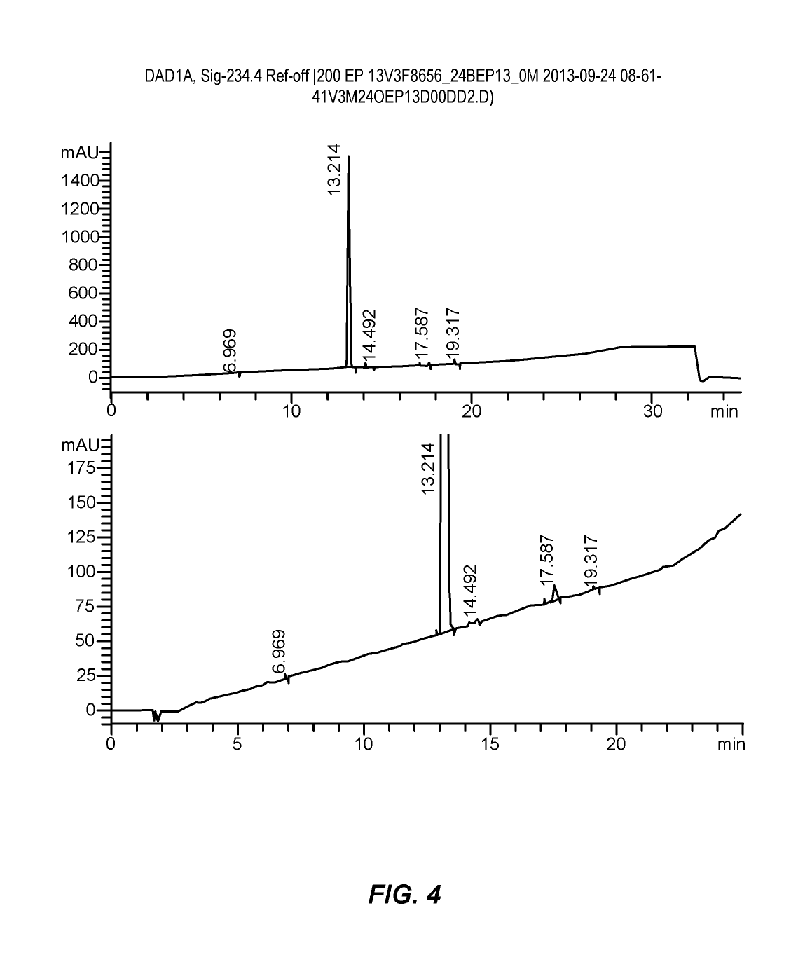

FIG. 4 shows an exemplary HPLC chromatogram of Form A.

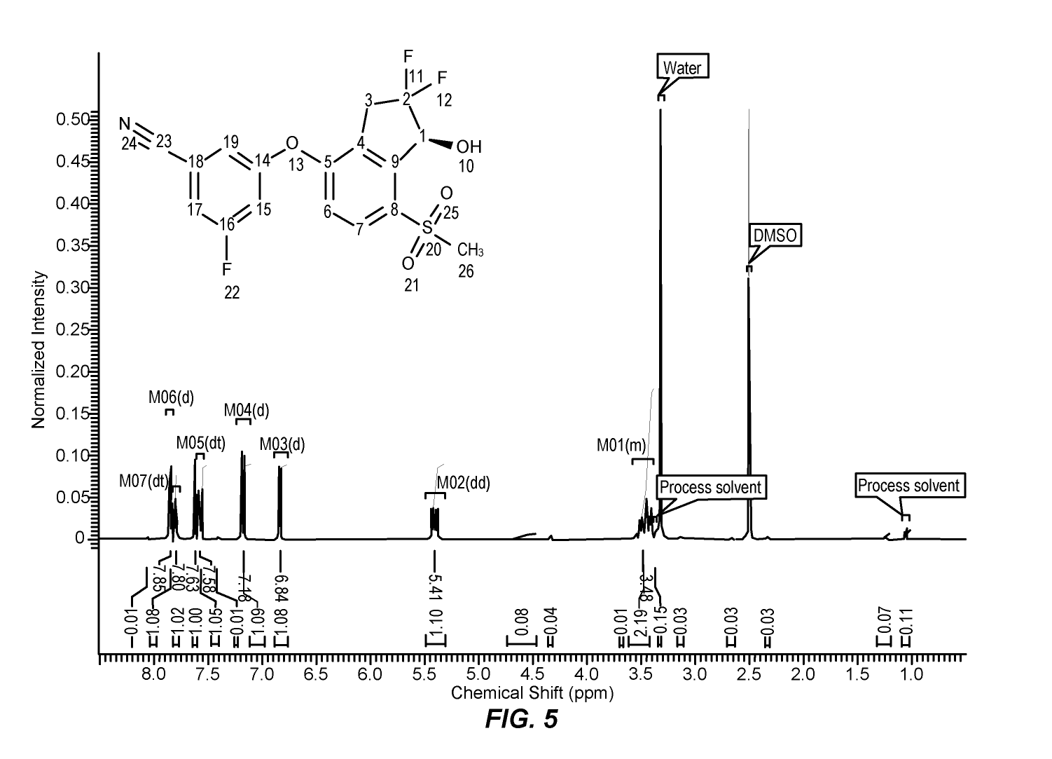

FIG. 5 shows an exemplary ¹H NMR spectrum of Form A.

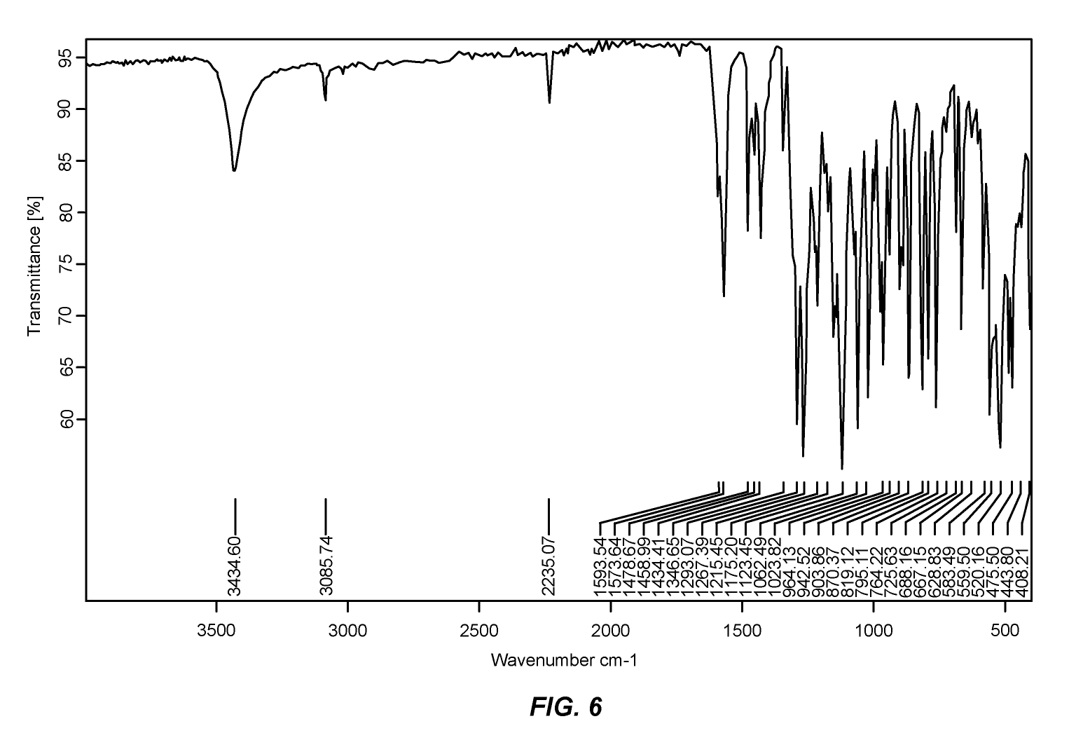

FIG. 6 shows an exemplary IR spectrum of Form A.

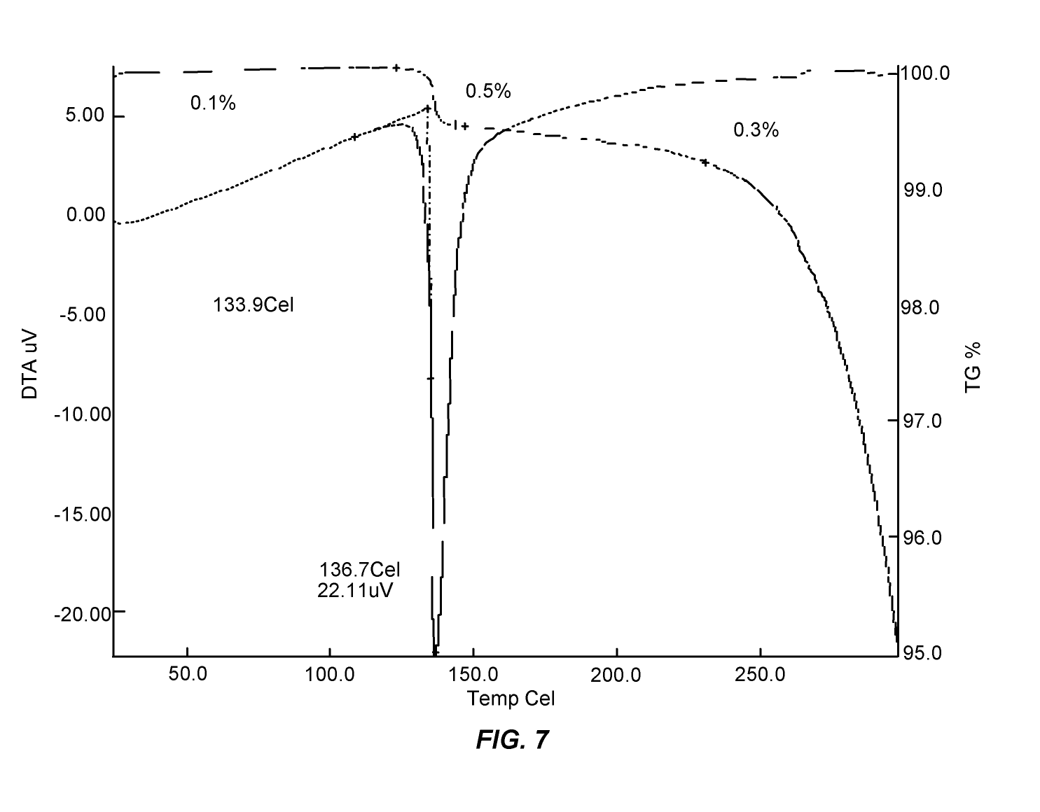

FIG. 7 shows an exemplary TG/DTA thermogram of Form A.

FIG. 8 shows an exemplary DSC thermogram of Form A.

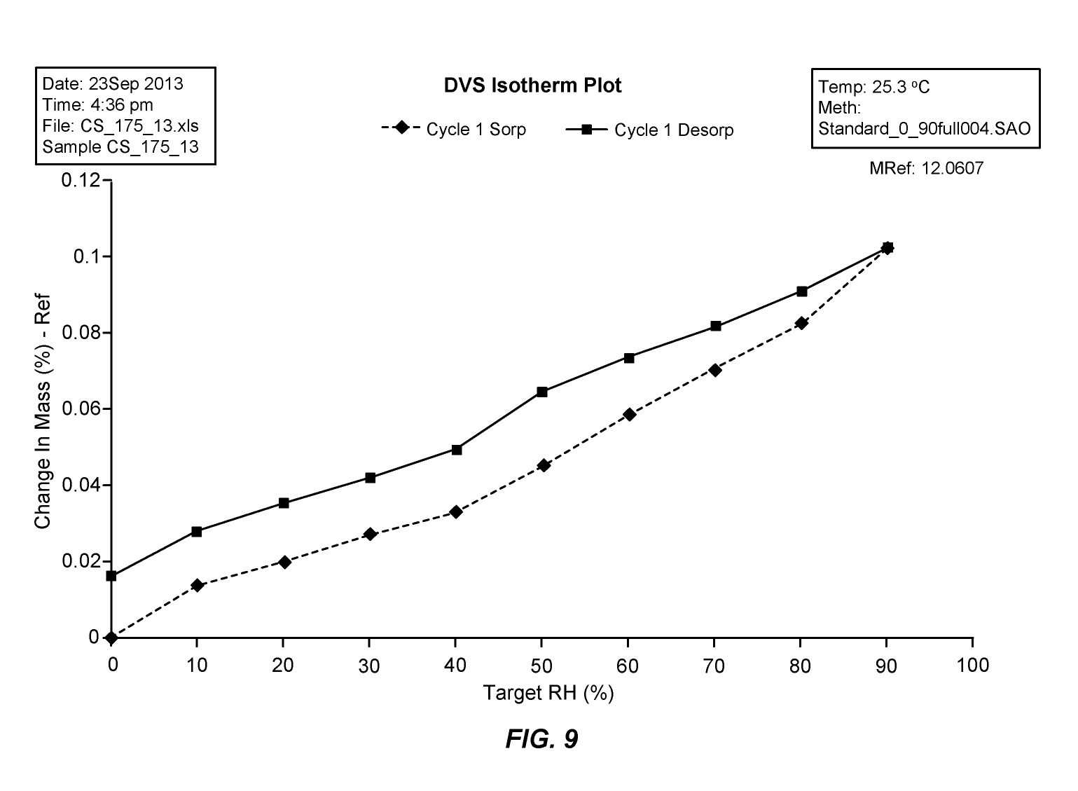

FIG. 9 shows an exemplary DVS isotherm plot of Form A.

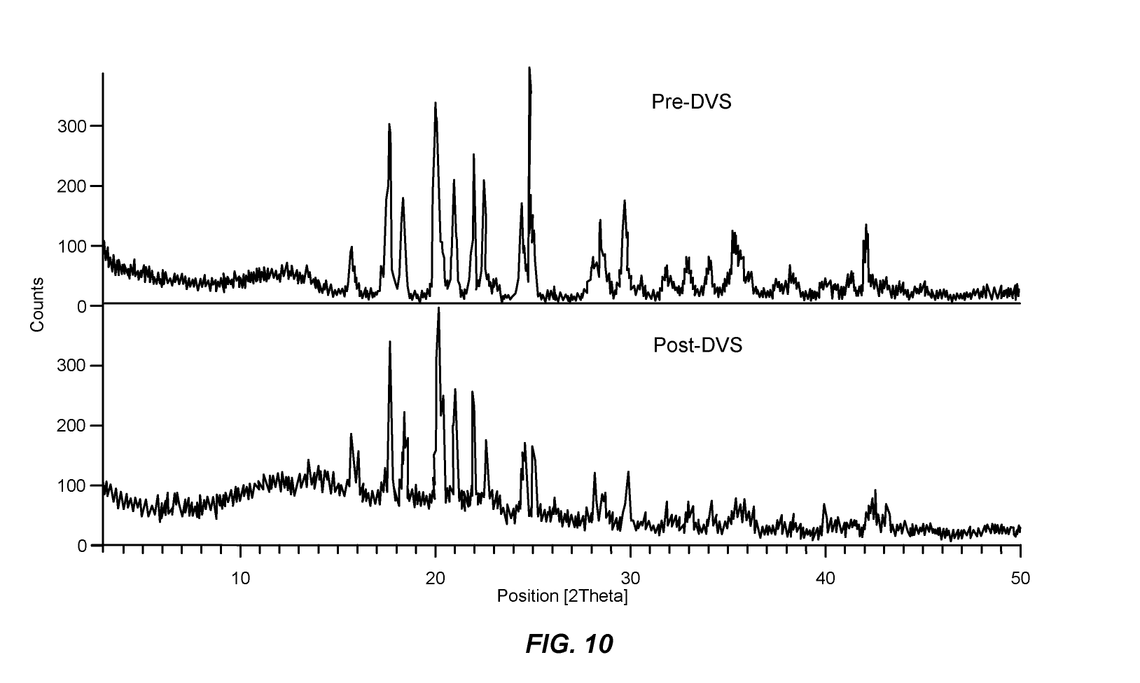

FIG. 10 shows comparison of XRPD diffractogram of Form A pre- and post-DVS Analysis.

In various embodiments, Form A may be obtained by crystallization from single solvent systems, including propanol and 2-butanol. In various embodiments, Form A may be obtained by crystallization from a binary solvent system comprising ethyl acetate and hexanes, as well as fast and slow cooling from binary solvent systems with dichloromethane as the anti-solvent. Form A may also be obtained from slurries in acetonitrile, ethanol, and isopropyl alcohol. In various embodiments, Form A is obtained by re-slurrying one or more non-A Forms in an anhydrous solvent.

In some embodiments, Form A is obtained by crystallizing a compound of Formula I with a chemical purity of less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%. In some embodiments. Form A is obtained by crystallizing a compound of Formula I with a chemical purity in the range of about 80% to about 96%, about 85% to about 96%, about 90% to about 96%, about 80% to 98%, about 85% to about 98%, about 90% to about 98%, about 92% to about 98%, about 94% to 98%, or about 96% to about 98%.

In some embodiments, the Form A is non-micronized. In some embodiments a majority of particles in the non-micronized polymorph Form A, for example greater than 60%, 70%, 80%, 90%, or 95% of particles in the polymorph Form A are smaller than 5 μm in diameter, 10 μm in diameter, 15 μm in diameter, 20 μm in diameter, 25 μm in diameter, 30 μm in diameter, 35 μm in diameter, 40 μm in diameter, 45 μm in diameter, 50 μm in diameter, 55 μm in diameter, 60 μm in diameter, 65 μm in diameter, 70 μm in diameter, 75 μm in diameter, 80 μm in diameter, 85 μm in diameter, 95 μm in diameter, 100 μm in diameter, 110 μm in diameter, 120 μm in diameter, 130 μm in diameter, 140 μm in diameter, 150 μm in diameter, 160 μm in diameter, 170 μm in diameter, 180 μm in diameter, 190 μm in diameter, 200 μm in diameter, 210 μm in diameter, 220 μm in diameter, 230 μm in diameter, 240 μm in diameter, 250 μm in diameter, 260 μm in diameter, 270 μm in diameter, 280 μm in diameter, 290 μm in diameter, or 300 μm in diameter. In some examples 60%, 70%, 80%, 90%, or 95% of the particles in non-micronized Form A have a diameter less than 100 μm.

In some embodiments, the Form A is micronized. In some embodiments a majority of particles in the non-micronized polymorph Form A, for example greater than 60%, 70%, 80%, 90%, or 95% of particles in the polymorph Form A are smaller than 5 μm in diameter, 10 μm in diameter, 15 μm in diameter, 20 μm in diameter, 25 μm in diameter, 30 μm in diameter, 35 μm in diameter, 40 μm in diameter, 45 μm in diameter, 50 μm in diameter, 55 μm in diameter, 60 μm in diameter, 65 μm in diameter, 70 μm in diameter, 75 μm in diameter, 80 μm in diameter, 85 μm in diameter, 95 μm in diameter, 100 μm in diameter, 110 μm in diameter, 120 μm in diameter, 130 μm in diameter, 140 μm in diameter, 150 μm in diameter, 160 μm in diameter, 170 μm in diameter, 180 μm in diameter, 190 μm in diameter, 200 μm in diameter, 210 μm in diameter, 220 μm in diameter, 230 μm in diameter, 240 μm in diameter, 250 μm in diameter, 260 μm in diameter, 270 μm in diameter, 280 μm in diameter, 290 μm in diameter, or 300 μm in diameter. In some examples 6004, 70%, 80%, 90%, or 95% of the particles in micronized Form A have a diameter less than 5 μm. In some examples 60%, 70%, 80%, 90%, or 95% of the particles in micronized Form A have a diameter less than 10 μm. In some examples 60%, 70%, 80%, 90%, or 95% of the particles in micronized Form A have a diameter less than 20 μm.

In some embodiments, the chemical purity of the polymorph Form A is greater than 60%, 70%, 80%, 90%, 95%, or 99%. In some embodiments, the purity of the polymorph Form A is greater than about 90%. In some embodiments, the purity of the polymorph Form A is greater than about 95%. In some embodiments, the chemical purity of the polymorph Form A greater than about 99%. The chemical purity of polymorph Form A may be measured by any available analytical technique, for example by HPLC analysis. In various embodiments, the enantiomeric purity of polymorph Form A is greater than about 90%, about 95%, or about 99%.

In various embodiments, the polymorph Form A is dry. In various embodiments, the polymorph Form A is non-solvated. In various embodiments, the polymorph Form A is non-hydrated. In various embodiments, the polymorph Form A is non-hygroscopic.

In various embodiments, the polymorph Form A shows about 0.01-10%, 0.03-1%, 0.05-1%, 0.07-1%, 0.1-1%, 0.2-1%, 0.3-1%, 0.4-1%, 0.5-1%, 0.6-1%, 0.6-1%, 0.7-1%, 0.8-1%, 0.9-1%, 0.01-0.9%, 0.03-0.9%, 0.05-0.9%, 0.07-0.9%, 0.1-0.9%, 0.2-0.9%, 0.3-0.9%, 0.4-0.9%, 0.5-0.9%, 0.6-0.9%, 0.6-0.9%, 0.7-0.9%, 0.8-0.9%, 0.01-0.8%, 0.03-0.8%, 0.05-0.8%, 0.07-0.8%, 0.1-0.8%, 0.2-0.8%, 0.3-0.8%, 0.4-0.8%, 0.5-0.8%, 0.6-0.8%, 0.6-0.8%, 0.7-0.8%, 0.01-0.7%, 0.03-0.7%, 0.05-0.7%, 0.07-0.7%, 0.1-0.7%, 0.2-0.7%, 0.3-0.7%, 0.4-0.7%, 0.5-0.7%, 0.6-0.7%, 0.6-0.7%, 0.01-0.6%, 0.03-0.6%, 0.05-0.6%, 0.07-0.6%, 0.1-0.6%, 0.2-0.6%, 0.3-0.6%, 0.4-0.6%, 0.5-0.6%, 0.01-0.5%, 0.03-0.5%, 0.05-0.5%, 0.07-0.5% 0, 0.1-0.5%, 0.2-0.5%, 0.3-0.5%, 0.4-0.5%, 0.5-0.5%, 0.01-0.4%, 0.03-0.4%, 0.05-0.4%, 0.07-0.4%, 0.1-0.4%, 0.2-0.4%, 0.3-0.4%, 0.01-0.3%, 0.03-0.3%, 0.05-0.3%, 0.07-0.3%, 0.1-0.3%, 0.2-0.3%, 0.01-0.2%, 0.03-0.2%, 0.05-0.2%, 0.07-0.2%, 0.1-0.2%, 0.01-0.1%, 0.03-0.1%, 0.05-0.1%, 0.07-0.1% weight loss at a temperature of about 100-150° C., for example at about 125-about 140° C. In various embodiments, the polymorph Form A shows about 0.05-0.5% weight loss at a temperature of about 125-about 140° C.

In various embodiments, the polymorph Form A is characterized by a single, sharp endotherm at about 130-135° C., for example at about 131-135° C., 132-135° C., 133-135° C., 134-135° C., 130-134° C., 131-134° C., 132-134° C., 133-134° C., 130-133° C., 131-133° C., 132-133° C., 130-132° C., 131-132° C. or 130-131° C. in the DTA trace. In various embodiments, the polymorph Form A is characterized by a single, sharp endotherm at about at about 133-134° C. in the DTA trace.

In various embodiments, the polymorph Form A decomposes above a temperature of about 100° C., about 150° C., about 200° C., about 250° C., about 300° C., about 350° C., about 400° C., about 450° C. or above 500° C. In some examples, the polymorph Form A decomposes above a temperature of about 250° C.

In some embodiments the polymorph Form A is insoluble in water. In some examples, the polymorph Form A has a solubility of less than about 1 μg/mL, 10 μg/mL, 20 μg/mL, 30 μg/mL, 40 μg/mL, 50 μg/mL, 60 μg/mL, 70 μg/mL, 80 μg/mL, 90 g/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL. In some embodiments the solubility of polymorph Form A in water is less than 2 mg/mL. In some embodiments the solubility of polymorph Form A in water is less than 5 mg/mL. In some embodiments the solubility of polymorph Form A in water is less than 10 mg/mL. In some embodiments the solubility of polymorph Form A in water is less than 20 mg/mL. In some embodiments the solubility of polymorph Form A in water is less than 30 mg/mL. In some embodiments the solubility of polymorph Form A in water is less than 40 mg/mL. In some embodiments the solubility of polymorph Form A in water is less than 50 mg/mL.

The composition of any one of the preceding claims, wherein the polymorph Form A is insoluble in solvents comprising diisopropyl ether, hexane, heptane, toluene or mixtures thereof. In some embodiments the solubility of polymorph Form A is one of these solvents is less than about 1 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL. In some embodiments the solubility of polymorph Form A in one of these solvents is less than 2 mg/mL. In some embodiments the solubility of polymorph Form A in one of these solvents is less than 5 mg/mL. In some embodiments the solubility of polymorph Form A in one of these solvents is less than 10 mg/mL. In some embodiments the solubility of polymorph Form A in one of these solvents is less than 20 mg/mL. In some embodiments the solubility of polymorph Form A in one of these solvents is less than 30 mg/mL. In some embodiments the solubility of polymorph Form A in one of these solvents is less than 40 mg/mL. In some embodiments the solubility of polymorph Form A in one of these solvents is less than 50 mg/mL.

In various embodiments, the polymorph Form A is soluble in polar aprotic solvents. In some examples, the solubility of the polymorph Form A in polar aprotic solvents is greater than about 50 mg/mL, about 100 mg/mL, about 150 mg/mL, about 200 mg/mL, about 250 mg/mL, about 300 mg/mL, about 400 mg/mL or about 500 mg/mL.

In some embodiments, the polymorph Form A is soluble in a solvent selected from a group consisting of acetone, acetone water mixture, acetonitrile, acetonitrile water mixture, dichloromethane, dimethylformamide, dimethylsulfoxide, 1,4-dioxane, ethanol, ethyl acetate, isopropyl acetate, methanol, methyl acetate, methylethyl ketone, methyl isobutyl ketone, N-methyl-2-pyrrolidone, tert-butyl methyl ether and THF. In various embodiments, the solubility of one of polymorph Form A in one of these solvents is greater than about 50 mg/mL, about 100 mg/mL, about 150 mg/mL, about 200 mg/mL, about 250 mg/mL, about 300 mg/mL, about 400 mg/mL or about 500 mg/mL. In various embodiments, the solubility of one of polymorph Form A in one of these solvents is greater than about 200 mg/mL.

In various embodiments, the polymorph Form A has a water content of about 0.1-about 1% as measured by KF titration. In some examples, the polymorph Form A has a water content of about 0.1-0.9%, 0.1-0.8%, 0.1-0.7%, 0.1-0.6%, 0.1-0.5%, 0.1-0.4%, 0.1-0.3%, 0.1-0.2%, 0.2-1.0%, 0.2-0.9%, 0.2-0.8%, 0.2-0.7%, 0.2-0.6%, 0.2-0.5%, 0.2-0.4%, 0.2-0.3%, 0.3-1.0%, 0.3-0.9%, 0.3-0.8%, 0.3-0.7%, 0.3-0.6%, 0.3-0.5%, 0.3-0.4%, 0.4-1.0%, 0.4-0.9%, 0.4-0.8%, 0.4-0.7%, 0.4-0.6%, 0.4-0.5%, 0.5-1.0%, 0.5-0.9%, 0.5-0.8%, 0.5-0.7%, 0.5-0.6%, 0.6-1.0%, 0.6-0.9%, 0.6-0.8%, 0.6-0.7%, 0.7-1.0%, 0.7-0.9%, 0.7-0.8%, 0.8-1.0% or 0.8-0.9% as measured by KF titration. In some examples, the polymorph Form A has a water content of about 0.5%, as measured by KF titration, before drying. In some examples, the polymorph Form A has a water content of about 0.09%, as measured by KF titration, after drying.

### Form B

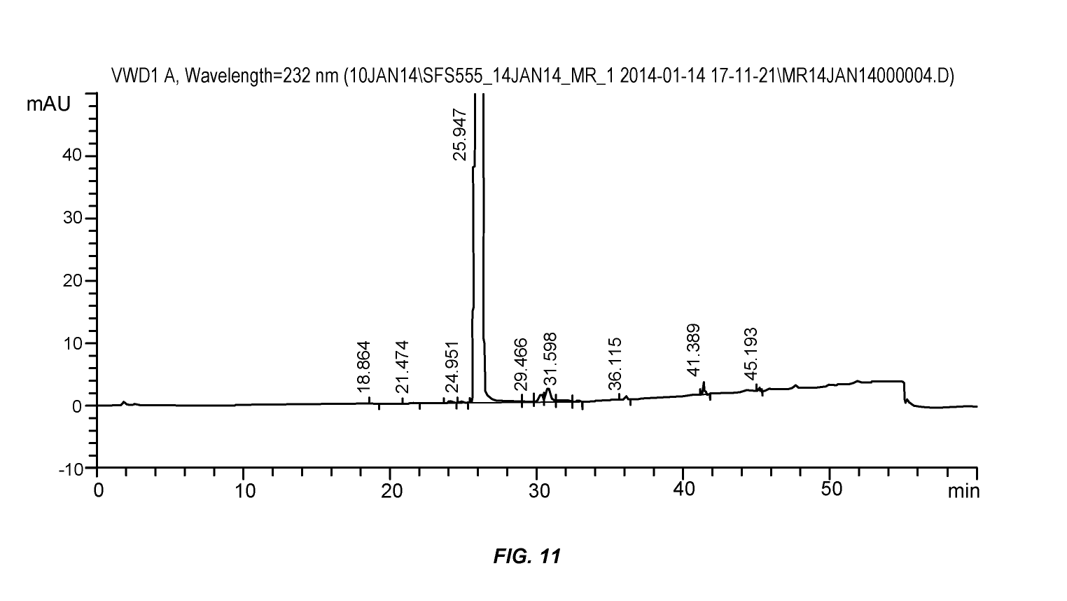

FIG. 11 shows an exemplary HPLC chromatogram of Form B.

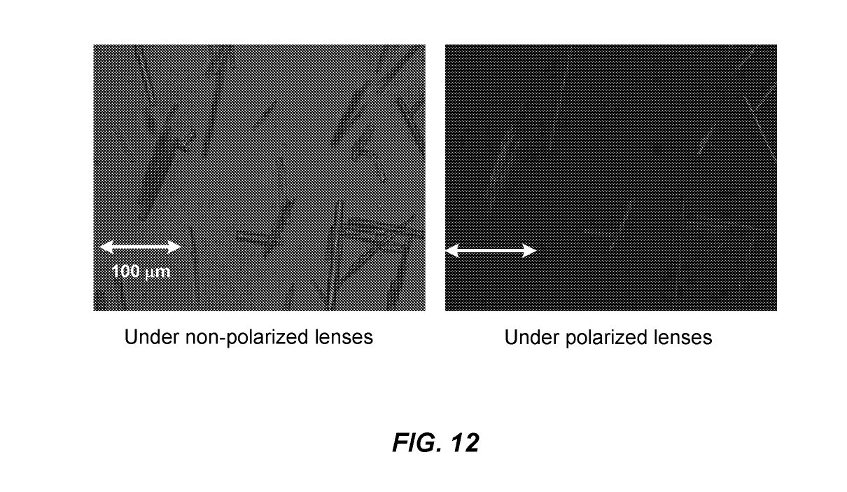

FIG. 12 shows exemplary PLM Images of Form B.

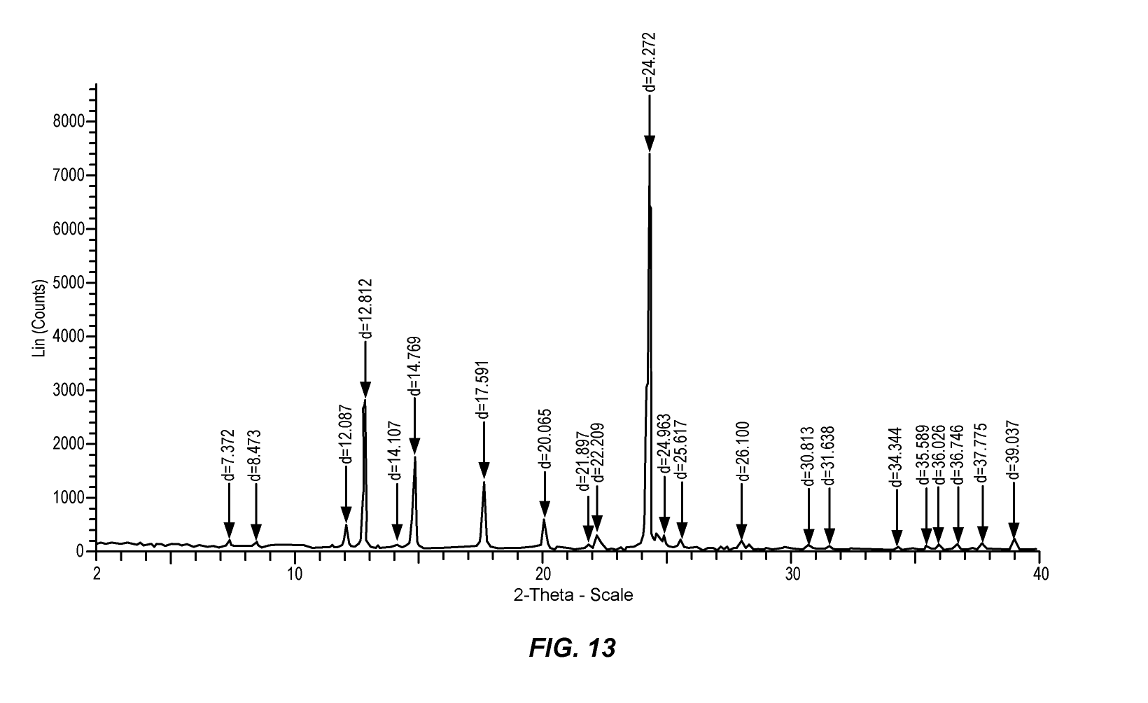

FIG. 13 shows the X-ray powder diffraction (XRPD) for Polymorph Form B.

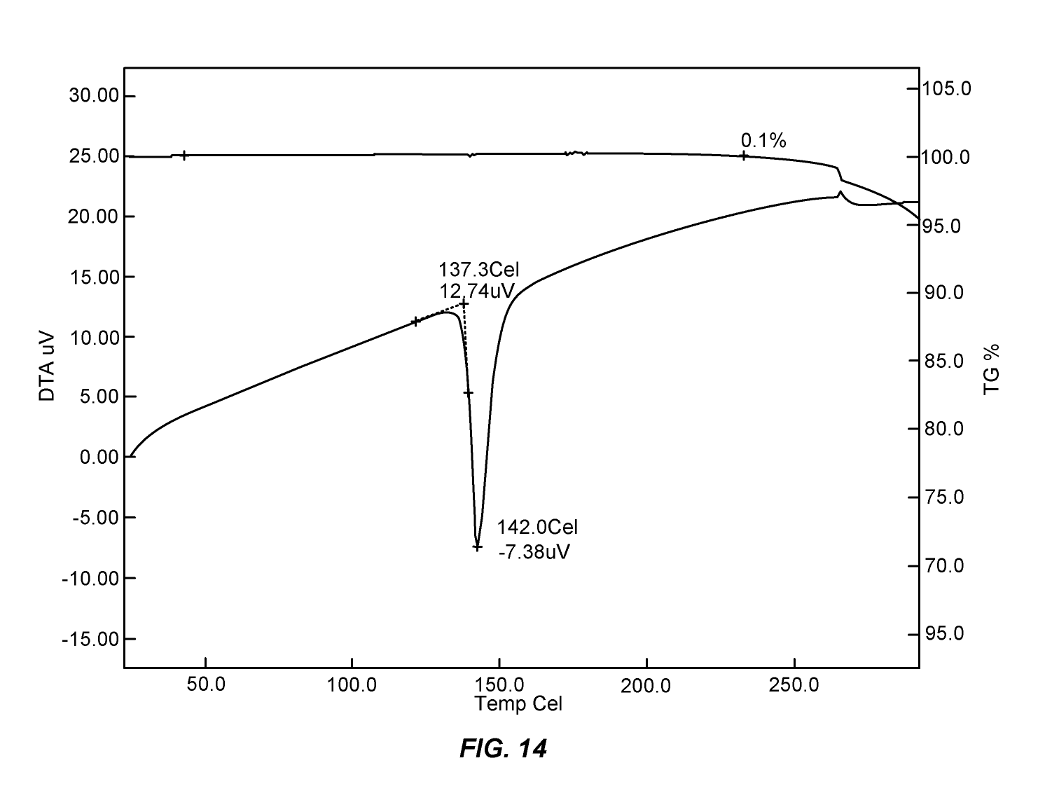

FIG. 14 shows an exemplary TG/DTA thermogram of Form B.

FIG. 15 shows an exemplary DSC thermogram of Form B.

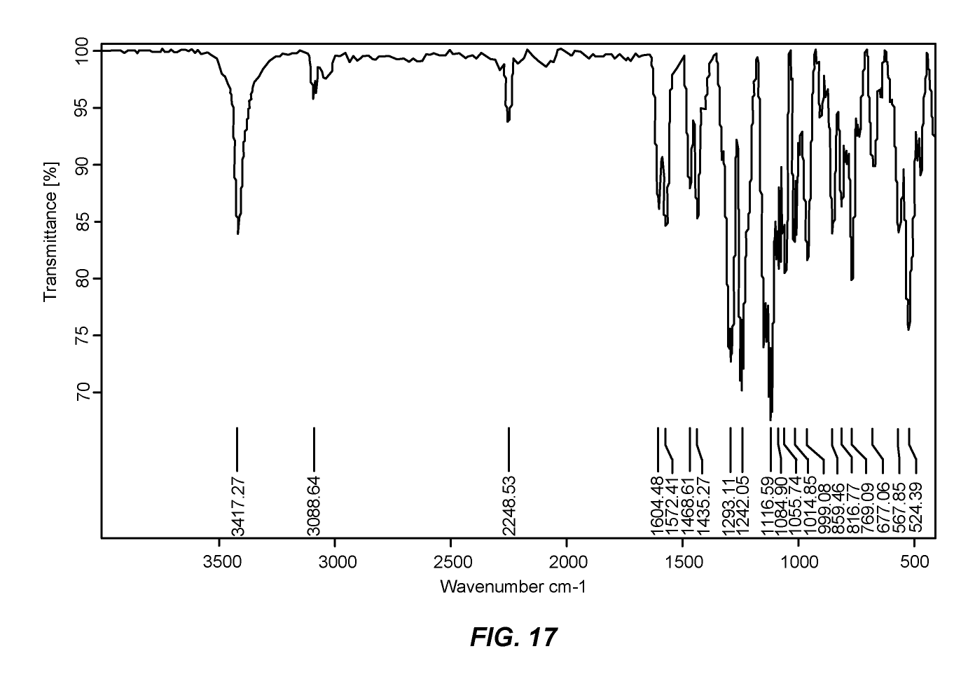

FIG. 17. shows an exemplary IR spectrum of Form B

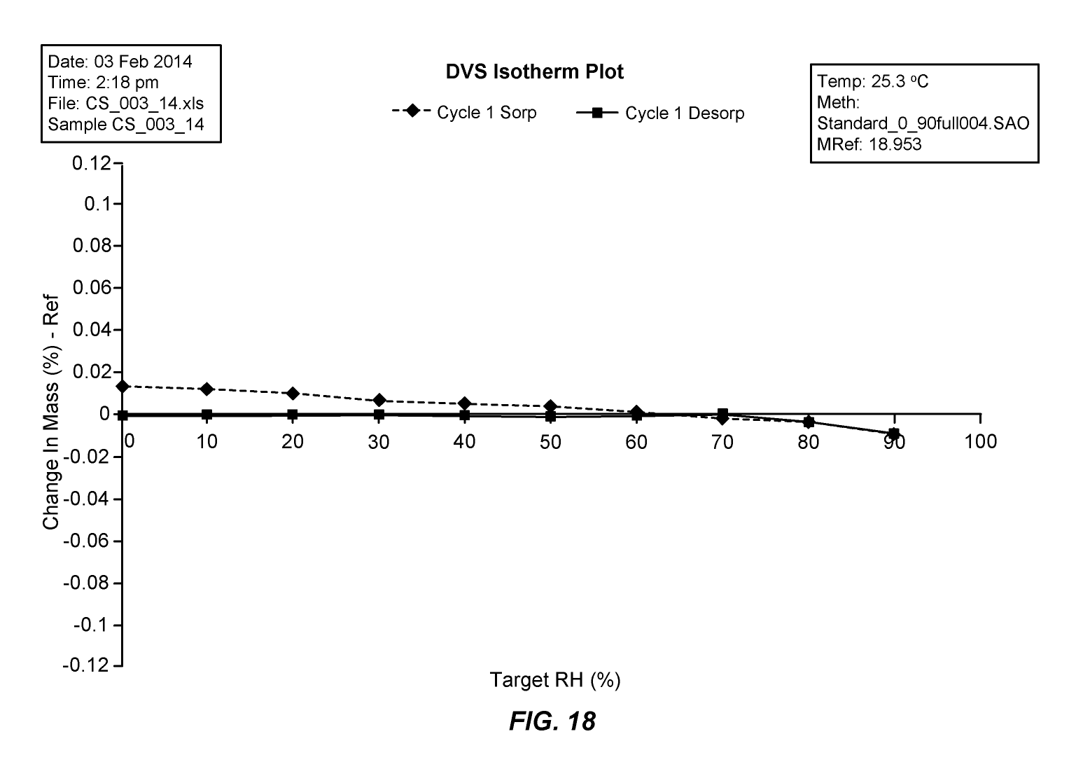

FIG. 18 shows an exemplary DVS isotherm plot of Form B.

In one embodiment, the polymorph according to the invention is Form B. FIG. 11 shows the XRPD for Polymorph Form B. The polymorph may be characterized by XRPD peaks at about 24.3 degrees 2θ. The polymorph may be characterized by XRPD peaks at 12.8, about 14.8, about 17.6 and about 24.3 degrees 2θ. The polymorph may be characterized by XRPD peaks at 12.8, about 14.8, about 17.6, about 20.1, about 20.9, about 22.2, about 24.3, about 25.0, about 25.6 and about 28.1 degrees 2θ.

FIG. 17 shows an exemplary DSC endotherm analysis for Form B. The symbol “exo” indicates an exotherm. In some embodiments, Form B is characterized by a DSC trace showing a peak at about 138° C. The peak may have an associated enthalpy of 25 mJ/mg

In various embodiments, the polymorph Form B is crystalline by polarized light microscopy (PLM). In some examples, the polymorph Form B may comprises thin rod or needle like crystals.

In some embodiments, the chemical purity of the polymorph Form B is greater than 60%, 70%, 80%, 90%, 95%, 99%, 99.6% or 99.9%. In some embodiments, the purity of the polymorph Form B is greater than about 90%. In some embodiments, the purity of the polymorph Form B is greater than about 95%. In some embodiments, the chemical purity of the polymorph Form B is greater than about 99%. The chemical purity of polymorph Form B may be measured by any available analytical technique, for example by HPLC analysis. In various embodiments, the enantiomeric purity of polymorph Form B is greater than about 90%, about 95%, about 990%, or about 99.9%.

In various embodiments, the polymorph Form B is dry. In various embodiments, the polymorph Form B is non-solvated. In various embodiments, the polymorph Form B is non-hydrated. In various embodiments, the polymorph Form B is non-hygroscopic.

In various embodiments, the polymorph Form B shows about 0.001-1% weight loss at a temperature of about 100-150° C., for example at about 125-about 140° C. In various embodiments, the polymorph Form B shows about 0.005-0.5%, 0.0.001-0.01%, 0.005-0.1% weight loss at a temperature of about 125-about 140° C.

In various embodiments, the polymorph Form B is characterized by a single, sharp endotherm at about 135-138° C., for example at about 135-136° C., 135-137° C., 135-138° C., 136-137° C., and 137-138° C. in the DTA trace. In various embodiments, the polymorph Form B is characterized by a single, sharp endotherm at about at about 137° C. in the TG/DTA trace. In various embodiments, the polymorph Form B is characterized by a single, sharp endotherm at about at about 138° C. in the TG/DTA trace.

In various embodiments, the polymorph Form B decomposes above a temperature of about 100° C., about 150° C., about 200° C., about 250° C., about 300° C., about 350° C., about 400° C., about 450° C., above 500° C., above 550° C., above 600° C., above 650° C., or about 700° C.

In some embodiments the polymorph Form B is insoluble in water. In some examples, the polymorph Form B has a solubility of less than about 1 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL. In some embodiments the solubility of polymorph Form B in water is less than 2 mg/mL. In some embodiments the solubility of polymorph Form B in water is less than 5 mg/mL. In some embodiments the solubility of polymorph Form B in water is less than 10 mg/mL. In some embodiments the solubility of polymorph Form B in water is less than 20 mg/mL. In some embodiments the solubility of polymorph Form B in water is less than 30 mg/mL. In some embodiments the solubility of polymorph Form B in water is less than 40 mg/mL. In some embodiments the solubility of polymorph Form B in water is less than 50 mg/mL.

The composition of any one of the preceding claims, wherein the polymorph Form B is insoluble in solvents comprising diisopropyl ether, hexane, heptane, toluene or mixtures thereof. In some embodiments the solubility of polymorph Form B is one of these solvents is less than about 1 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL. In some embodiments the solubility of polymorph Form B in one of these solvents is less than 2 mg/mL. In some embodiments the solubility of polymorph Form B in one of these solvents is less than 5 mg/mL. In some embodiments the solubility of polymorph Form B in one of these solvents is less than 10 mg/mL. In some embodiments the solubility of polymorph Form B in one of these solvents is less than 20 mg/mL. In some embodiments the solubility of polymorph Form B in one of these solvents is less than 30 mg/mL. In some embodiments the solubility of polymorph Form B in one of these solvents is less than 40 mg/mL. In some embodiments the solubility of polymorph Form B in one of these solvents is less than 50 mg/mL.

In various embodiments, the polymorph Form B is soluble in polar aprotic solvents. In some examples, the solubility of the polymorph Form B in polar aprotic solvents is greater than about 50 mg/mL, about 100 mg/mL. about 150 mg/mL, about 200 mg/mL, about 250 mg/mL, about 300 mg/mL, about 400 mg/mL or about 500 mg/mL.

In some embodiments, the polymorph Form B is soluble in a solvent selected from a group consisting of acetone, acetone water mixture, acetonitrile, acetonitrile water mixture, dichloromethane, dimethylformamide, dimethylsulfoxide, 1,4-dioxane, ethanol, ethyl acetate, isopropyl acetate, methanol, methyl acetate, methylethyl ketone, methyl isobutyl ketone, N-methyl-2-pyrrolidone, tert-butyl methyl ether and THF. In various embodiments, the solubility of one of polymorph Form B in one of these solvents is greater than about 50 mg/mL, about 100 mg/mL, about 150 mg/mL, about 200 mg/mL, about 250 mg/mL, about 300 mg/mL, about 400 mg/mL or about 500 mg/mL. In various embodiments, the solubility of one of polymorph Form B in one of these solvents is greater than about 200 mg/mL.

In various embodiments, the polymorph Form B has a water content of about 0.1-about 1% as measured by KF titration. In some examples, the polymorph Form B has a water content of about 0.1-0.9%, 0.1-0.8%, 0.1-0.7%, 0.1-0.6%, 0.1-0.5%, 0.1-0.4%, 0.1-0.3%, 0.1-0.2%, 0.2-1.0%, 0.2-0.9%, 0.2-0.8%, 0.2-0.7%, 0.2-0.6%, 0.2-0.5%, 0.2-0.4%, 0.2-0.3%, 0.3-1.0%, 0.3-0.9%, 0.3-0.8%, 0.3-0.7%, 0.3-0.6%, 0.3-0.5%, 0.3-0.4%, 0.4-1.0%, 0.4-0.9%, 0.4-0.8%, 0.4-0.7%, 0.4-0.6%, 0.4-0.5%, 0.5-1.0%, 0.5-0.9%, 0.5-0.8%, 0.5-0.7%, 0.5-0.6%, 0.6-1.0%, 0.6-0.9%, 0.6-0.8%, 0.6-0.7%, 0.7-1.0%, 0.7-0.9%, 0.7-0.8%, 0.8-1.0% or 0.8-0.9% as measured by KF titration. In some examples, the polymorph Form B has a water content of about 0.1%, as measured by KF titration. In some examples, the polymorph Form B has a water content of about 0.2%, as measured by KF titration. In some examples, the polymorph Form B has a water content of about 0.3%, as measured by KF titration.

In various embodiments, Form B is obtained in a mixture with non-B polymorph forms. For example, in various embodiments, Form B is present as a composition further comprising one or more non-B polymorph forms. The amount of non-B polymorph forms may vary. For example, in various embodiments, the weight ratio of polymorph Form B to the total amount of one or more non-B polymorphs is greater than about 1:1, greater than about 2:1, greater than about 3:1, greater than about 4:1, greater than about 5:1, greater than about 6:1 greater than about 7:1, greater than about 8:1, greater than about 9:1, greater than about 9.5:1, or greater than about 99:1. Similarly, when formulated in pharmaceutical compositions, various amounts of non-B polymorph form may be present. In various embodiments the weight ratio of polymorph Form B to the total amount of one or more non-B polymorphs in a pharmaceutical composition is greater than about 5:1, greater than about 6:1, greater than about 7:1, greater than about 8:1, greater than about 9:1, greater than about 9.5:1, or greater than about 99:1.

In various embodiments, Form B is obtained from direct workup of the synthetic step producing the compound of Formula I, and non-B Forms are not obtained, or are obtained as a minority component. In some embodiments, Form B is obtained by recrystallization of compound of Formula I. In some examples, the recrystallization process includes complete dissolution of the compound of Formula I followed by optional filtration to remove any insoluble particles, and subsequent crystallization to yield Form B. In some embodiments, complete dissolution and filtration may not performed, in which case a slurry is formed which converts to Form B without complete dissolution of the compound of Formula 1. In some examples, the complete dissolution of the compound of Formula I is performed at a temperature above the ambient temperature. In some examples, the complete dissolution of the compound of Formula I is performed at a temperature of about 30-100° C., for example at about 30-90° C., about 30-80° C., about 30-70° C., about 30-60° C., about 30-50° C., about 30-40° C., about 40-100° C., about 40-90° C., about 40-80° C., about 40-70° C., about 40-60° C., about 40-50° C., about 50-100° C., about 50-90° C., about 50-80° C., about 50-70° C., about 50-60° C., about 60-100° C., about 60-90° C., about 60-80° C., about 60-70° C., about 70-100° C., 70-90° C., 70-80° C., 80-100° C., 80-90° C. or 90-100° C. In some examples, the complete dissolution of the compound of Formula I is performed at a temperature of about 70-85° C., for example 70-72° C. In some examples, the recrystallization solvent is 2-propanol and the complete dissolution of the compound of Formula I is performed at a temperature of about 70-72° C. In some examples, subsequent crystallization (after complete dissolution) is carried at ambient temperature for a time period of about 1-24 h. In some examples, subsequent crystallization is carried at a temperature below the ambient temperature for a time period of about 1-24 h. In some examples, subsequent crystallization is carried at a temperature of about 0-25° C. for a time period of about 1-24 h, for example for about 1-20 h, 1-15 h, 1-10 h, 1-5 g, 5-24 h, 5-20 h, 5-15 h, 5-10 h, 10-24 h, 10-20 h, 10-15 h, 20-24 h. In some examples, subsequent crystallization is carried at a temperature of about 5-10° C. for a time period of about 1-24 h, for example for about 1-20 h, 1-15 h, 1-10 h, 1-5 g, 5-24 h, 5-20 h, 5-15 h, 5-10 h, 10-24 h, 10-20 h, 10-15 h, 20-24 h.

In some embodiments, Form B is obtained by crystallizing a compound of Formula I with a chemical purity of greater than about 98%, greater than about 99%, or greater than about 99.5%. In some embodiments, Form B is obtained by crystallizing a compound of Formula I with a chemical purity in the range of about 98% to about 98.5%, about 98% to about 99%, or about 98% to about 99.5%.

### Amorphous Form

Amorphous Form may alternatively be made by dissolution of a crystalline form followed by removal of solvent under conditions in which stable crystals are not formed. For example, solidification may occur by rapid removal of solvent, by rapid addition of an anti-solvent (causing the amorphous form to crash out of solution), crash cooling at −20° C., temperature recycling from room temperature to about 40° C., or by physical interruption of the crystallization process.

In various embodiments, the amorphous form can be obtained by fast cooling from single solvent crystallization systems, including methyl isobutyl ketone, N-methyl 2-pyrrolidone and toluene. In various embodiments, the amorphous form can be obtained by slow cooling from single solvent crystallization systems, including methyl isobutyl ketone, N-methyl 2-pyrrolidone and toluene. In various embodiments, amorphous form may be obtained by fast cooling crystallization from binary solvent systems, methyl isobutyl ketone, N-methyl 2-pyrrolidone and toluene as the primary solvent. The amorphous form may be characterized by lack of any significant peaks in XRPD spectrum.

### III. Compositions

The invention provides compositions, including pharmaceutical compositions, comprising one or more polymorphs of the present invention.

In various embodiments, the ratio of desired polymorph such as Form B to all other polymorphs may be greater than about 1:1, 2:1, 3:1, 4:1, 5:1, 6:1, 7:1, 8:1, 9:1, or more.

In various embodiments, the ratio of desired polymorph Form A to all other polymorphs may be greater than about 1:1, 2:1, 3:1, 4:1, 5:1, 6:1, 7:1, 8:1, 9:1, or more.

In various embodiments, the ratio of the desired anhydrous polymorph to all other polymorphs may be greater than about 1:1, 2:1, 3:1, 4:1, 5:1, 6:1, 7:1, 8:1, 9:1, or more.

The subject pharmaceutical compositions are typically formulated to provide a therapeutically effective amount of a polymorph of the present invention as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the subject polymorphs and other agent(s) may be mixed into a preparation or both components may be formulated into separate preparations to use them in combination separately or at the same time.

In some embodiments, the concentration of one or more of the polymorphs provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the polymorphs in the pharmaceutical compositions of the present invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25%, 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the polymorphs in the pharmaceutical compositions of the present invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the amount of one or more of the polymorphs in 1 mL of the pharmaceutical compositions of the present invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the polymorphs in 1 mL of the pharmaceutical compositions of the present invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the polymorphs of the present invention in 1 mL of the pharmaceutical compositions is in the range of 0.0001-10 g, 0.0005-9 g, 0.001-8 g, 0.005-7 g, 0.01-6 g, 0.05-5 g, 0.1-4 g, 0.5-4 g, or 1-3 g.

The polymorphs according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 100 to 2000 mg per day. The exact dosage will depend upon the route of administration, the form in which the polymorphs is administered, the subject to be treated, the body weight of the subject to be treated, and the preference and experience of the attending physician.

Described below are non-limiting exemplary pharmaceutical compositions and methods for preparing the same.

Pharmaceutical compositions for oral administration: In some embodiments, the invention provides a pharmaceutical composition for oral administration containing a polymorph of the present invention, and a pharmaceutical excipient suitable for oral administration.

In some embodiments, the invention provides a solid pharmaceutical composition for oral administration containing: (i) an effective amount of a compound of the present invention; optionally (ii) an effective amount of a second agent; and (iii) one or more pharmaceutical excipients suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the polymorphs disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

In some cases, colloid particles include at least one cationic agent and at least one non-ionic surfactant such as a poloxamer, tyloxapol, a polysorbate, a polyoxyethylene castor oil derivative, a sorbitan ester, or a polyoxyl stearate. In some cases, the cationic agent is an alkylamine, a tertiary alkyl amine, a quaternary ammonium compound, a cationic lipid, an amino alcohol, a biguanidine salt, a cationic compound or a mixture thereof. In some cases the cationic agent is a biguanidine salt such as chlorhexidine, polyaminopropyl biguanidine, phenformin, alkylbiguanidine, or a mixture thereof. In some cases, the quaternary ammonium compound is a benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetalkonium halide, cetethyldimonium halide, cetylpyridinium halide, benzododecinium halide, chlorallyl methenamine halide, rnyristylalkonium halide, stearalkonium halide or a mixture of two or more thereof. In some cases, cationic agent is a benzalkonium chloride, lauralkonium chloride, benzododecinium bromide, benzethenium chloride, hexadecyltrimethylammonium bromide, tetradecyltrimethylammonium bromide, dodecyltrimethylammonium bromide or a mixture of two or more thereof. In some cases, the oil phase is mineral oil and light mineral oil, medium chain triglycerides (MCT), coconut oil; hydrogenated oils comprising hydrogenated cottonseed oil, hydrogenated palm oil, hydrogenate castor oil or hydrogenated soybean oil; polyoxyethylene hydrogenated castor oil derivatives comprising poluoxyl-40 hydrogenated castor oil, polyoxyl-60 hydrogenated castor oil or polyoxyl-100 hydrogenated castor oil.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropyl alcohol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone, esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water. In various embodiments, a solubilizer comprising polyglycol mono- and di-esters of 12-hydroxystearic acid and about 30% free polyethylene glycol (available as Solutol HS 15) is used as a solubilizer.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical compositions for injection. In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions for topical (e.g., transdermal) delivery. In some embodiments, the invention provides a pharmaceutical composition for transdermal delivery containing a compound of the present invention and at least one pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

In some embodiments, the invention provides a pharmaceutical composition for treating ophthalmic disorders. The composition is formulated for ocular administration and it contains an effective amount of one or more polymorphs of the present invention and a pharmaceutical excipient suitable for ocular administration. Pharmaceutical compositions of the invention suitable for ocular administration can be presented as discrete dosage forms, such as drops or sprays each containing a predetermined amount of an active ingredient in a solution, or a suspension in an aqueous or non-aqueous liquid, an oil-in-water emulsion, or a water-in-oil liquid emulsion. Eye drops may be prepared by dissolving the active ingredient in a sterile aqueous solution such as physiological saline, buffering solution, etc., or by combining powder compositions to be dissolved before use. Other vehicles may be chosen, as is known in the art, including but not limited to: balance salt solution, saline solution, water soluble polyethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

Other pharmaceutical compositions. Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

Administration of the polymorphs or pharmaceutical composition of the present invention can be effected by any method that enables delivery of the polymorphs to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Polymorphs can also be administered intraadiposally or intrathecally.

The amount of the compound administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose. Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the agents of the invention may continue as long as necessary. In some embodiments, an agent of the invention is administered for more than 1, 2, 3, 4, 5, 6, 7, 14, or 28 days or longer. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, polymorphs of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Polymorphs of the invention may be applied to the surface of the stent by various methods such as dip/spin coating, spray coating, dip-coating, and/or brush-coating. The polymorphs may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, polymorphs of the invention may be covalently linked to a stent or graft. A covalent linker may be used which degrades in vivo, leading to the release of the compound of the invention. Any bio-labile linkage may be used for such a purpose, such as ester, amide or anhydride linkages. Polymorphs of the invention may additionally be administered intravascularly from a balloon used during angioplasty. Extravascular administration of the polymorphs via the pericardia or via advential application of formulations of the invention may also be performed to decrease restenosis.

A variety of stent devices which may be used as described are disclosed, for example, in the following references, all of which are hereby incorporated by reference: U.S. Pat. No. 5,451,233; U.S. Pat. No. 5,040,548; U.S. Pat. No. 5,061,273; U.S. Pat. No. 5,496,346; U.S. Pat. No. 5,292,331; U.S. Pat. No. 5,674,278; U.S. Pat. No. 3,657,744; U.S. Pat. No. 4,739,762; U.S. Pat. No. 5,195,984; U.S. Pat. No. 5,292,331; U.S. Pat. No. 5,674,278; U.S. Pat. No. 5,879,382; U.S. Pat. No. 6,344,053.

The polymorphs of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

The invention also provides kits. The kits include a compound or polymorphs of the present invention as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The polymorphs described herein can be used in combination with the agents disclosed herein or other suitable agents, depending on the condition being treated. Hence, in some embodiments the polymorphs of the invention will be co-administered with other agents as described above. When used in combination therapy, the polymorphs described herein may be administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the present invention and any of the agents described above can be simultaneously administered, wherein both the agents are present in separate formulations. In another alternative, a compound of the present invention can be administered just followed by and any of the agents described above, or vice versa. In the separate administration protocol, a compound of the present invention and any of the agents described above may be administered a few minutes apart, or a few hours apart, or a few days apart.

### IV. Methods of Treatment

In one aspect, the present invention provides a method for treating a proliferative disorder in a subject in need thereof, comprising administering to said subject a polymorph of Formula I disclosed herein. In some embodiments, the proliferative disorder is a cancer condition. In some further embodiments, said cancer condition is a cancer selected from the group consisting of lung cancer, head and neck squamous cell carcinoma, pancreatic cancer, breast cancer, ovarian cancer, renal cell carcinoma, prostate cancer, neuroendocrine cancer, gastric cancer, bladder cancer and colon cancer. In another embodiment, the cancer condition is renal cell carcinoma. In some embodiments, the polymorph is Form A. In some embodiments, the polymorph is Form B. In some embodiments, the polymorph is the amorphous polymorph of Formula I.

In a further embodiment, the present invention provides a method of treating a cancer condition, wherein a polymorph of Formula I is effective in one or more of inhibiting proliferation of cancer cells, inhibiting metastasis of cancer cells, killing cancer cells and reducing severity or incidence of symptoms associated with the presence of cancer cells. In some other embodiments, said method comprises administering to the cancer cells a therapeutically effective amount of a polymorph of Formula I disclosed herein. In some embodiments, the administration takes place in vitro. In other embodiments, the administration takes place in vivo. In some embodiments, the polymorph is Form A. In some embodiments, the polymorph is Form B. In some embodiments, the polymorph is the amorphous polymorph of Formula I.

As used herein, a therapeutically effective amount of a polymorph of Formula I refers to an amount sufficient to effect the intended application, including but not limited to, disease treatment, as defined herein. Also contemplated in the subject methods is the use of a sub-therapeutic amount of a polymorph of Formula I for treating an intended disease condition. In some embodiments, the polymorph is Form A. In some embodiments, the polymorph is Form B. In some embodiments, the polymorph is the amorphous polymorph of Formula I.

The amount of the Formula I polymorph administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

Measuring inhibition of biological effects of Formula I polymorph can comprise performing an assay on a biological sample, such as a sample from a subject. Any of a variety of samples may be selected, depending on the assay. Examples of samples include, but are not limited to blood samples (e.g. blood plasma or serum), exhaled breath condensate samples, bronchoalveolar lavage fluid, sputum samples, urine samples, and tissue samples.

A subject being treated with a Formula I polymorph may be monitored to determine the effectiveness of treatment, and the treatment regimen may be adjusted based on the subject's physiological response to treatment. For example, if inhibition of a biological effect of HIF-2α inhibition is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with a HIF-2α inhibitor is discontinued if inhibition of the biological effect is above or below a threshold, such as in a lack of response or an adverse reaction. The biological effect may be a change in any of a variety of physiological indicators.

In some embodiments, the polymorphs of Formula I are HIF-2α inhibitor. In general, a HIF-2α inhibitor is a compound that inhibits one or more biological effects of HIF-2α. Examples of biological effects of HIF-2α include, but are not limited to, heterodimerization of HIF-2α to HIF-1β, HIF-2α target gene expression, VEGF gene expression, and VEGF protein secretion. In some embodiments, the HIF-2α inhibitor is selective for HIF-2α, such that the inhibitor inhibits heterodimerization of HIF-2α to HIF-1β but not heterodimerization of HIF-1α to HIF-1β. Such biological effects may be inhibited by about or more than about 10%, 20%, 30%, 40%, 50%, 60%, 70%, 80%, 90%, 95%, or more.

Hypoxia-inducible factors (HIFs), like HIF-2α, are transcription factors that respond to changes in available oxygen in the cellular environment (e.g. a decrease in oxygen, or hypoxia). The HIF signaling cascade mediates the effects of hypoxia, the state of low oxygen concentration, on the cell. Hypoxia often keeps cells from differentiating. However, hypoxia promotes the formation of blood vessels, and is important for the formation of a vascular system in embryos, and cancer tumors. The hypoxia in wounds also promotes the migration of keratinocytes and the restoration of the epithelium. A HIF-2α inhibitor of the present disclosure may be administered in an amount effective in reducing any one or more of such effects of HIF-2α activity.

HIF-2α activity can be inhibited by inhibiting heterodimerization of HIF-2α to HIF-1β (ARNT), such as with inhibitor compounds disclosed herein. A variety of methods for measuring HIF-2α dimerization are available. In some embodiments, the HIF-2α inhibitor binds the PAS-B domain cavity of HIF-2α.

Inhibition of heterodimerization of HIF-2α to HIF-1β (ARNT) may also be determined by a reduction in HIF-2α target gene mRNA expression. mRNA quantitation can be performed using real-time PCR technology. (Wong, et al, “Real-time PCR for mRNA quantitation”, 2005. BioTechniques 39, 1:1-1.). Yet another method for determining inhibition of heterodimerization of HIF-2α to HIF-1β (ARNT) is by co-immunoprecipitation.

As described herein, HIF-2α is a transcription factor that plays important roles in regulating expression of target genes. Non-limiting examples of HIF-2α target genes include HMOX1, SFTPA1, CXCR4, PAI1, BDNF, hTERT, ATP7A, and VEGF. For instance, HIF-2α is an activator of VEGF. A HIF-2α inhibitor of the present disclosure may be administered in an amount effective in reducing expression of any one or more of genes induced by HIF-2α activity. A variety of methods is available for the detection of gene expression levels, and includes the detection of gene transcription products (polynucleotides) and translation products (polypeptides). For example, gene expression can be detected and quantified at the DNA, RNA or mRNA level. Various methods that have been used to quantify mRNA include in situ hybridization techniques, fluorescent in situ hybridization techniques, reporter genes, RNase protection assays, Northern blotting, reverse transcription (RT)-PCR, SAGE, DNA microarray, tiling array, and RNA-seq. Examples of methods for the detection of polynucleotides include, but are not limited to selective colorimetric detection of polynucleotides based on the distance-dependent optical properties of gold nanoparticles, and solution phase detection of polynucleotides using interacting fluorescent labels and competitive hybridization. Examples for the detection of proteins include, but are not limited to microscopy and protein immunostaining, protein immunoprecipitation, immunoelectrophoresis, western blot, BCA assay, spectrophotometry, mass spectrophotometry and enzyme assay.

In some embodiments, inhibition of HIF-2α is characterized by a decrease in VEGF gene expression. The decrease may be measured by any of a variety of methods, such as those described herein. As a further example, the mRNA expression level of VEGF can be measured by quantitative PCR (QT-PCR), microarray, RNA-seq and nanostring. As another example, an ELISA assay can be used to measure the level VEGF protein secretion.

In some other embodiments, the subject methods are useful for treating a disease condition associated with HIF-2α. Any disease condition that results directly or indirectly from an abnormal activity or expression level of HIF-2α can be an intended disease condition. In some embodiments, the disease condition is a proliferative disorder, such as described herein, including but not limited to cancer. A role of HIF-2α in tumorigenesis and tumor progression has been implicated in many human cancers. Constitutively active HIF-2α may be the result of defective VHL or a low concentration of oxygen in a cancer cell. Rapidly growing tumors are normally hypoxic due to poor vascularization, a condition that activates HIF-2α in support of tumor cell survival and proliferation. Constitutive activation of HIF-2α is emerging as a common theme in diverse human cancers, consequently agents that target HIF-2α have therapeutic value.

The data presented in the Examples herein below demonstrate the anti-cancer effects of a HIF-2α inhibitor. As such, the subject method is particularly useful for treating a proliferative disorder, such as a neoplastic condition. Non-limiting examples of such conditions include but are not limited to acanthoma, acinic cell carcinoma, acoustic neuroma, acral lentiginous melanoma, acrospiroma, acute eosinophilic leukemia, acute lymphoblastic leukemia, acute megakaryoblastic leukemia, acute monocytic leukemia, acute myeloblastic leukemia with maturation, acute myeloid dendritic cell leukemia, acute myeloid leukemia, acute promyelocytic leukemia, adamantinoma, adenocarcinoma, adenoid cystic carcinoma, adenoma, adenomatoid odontogenic tumor, adrenocortical carcinoma, adult T-cell leukemia, aggressive NK-cell leukemia, AIDS-related cancers, AIDS-related lymphoma, alveolar soft part sarcoma, ameloblastic fibroma, anal cancer, anaplastic large cell lymphoma, anaplastic thyroid cancer, angioimmunoblastic T-cell lymphoma, angiomyolipoma, angiosarcoma, appendix cancer, astrocytoma, atypical teratoid rhabdoid tumor, basal cell carcinoma, basal-like carcinoma, B-cell leukemia, B-cell lymphoma, bellini duct carcinoma, biliary tract cancer, bladder cancer, blastoma, bone cancer, bone tumor, brain stem glioma, brain tumor, breast cancer, brenner tumor, bronchial tumor, bronchioloalveolar carcinoma, brown tumor, Burkitt's lymphoma, carcinoid tumor, carcinoma, carcinosarcoma, Castleman's disease, central nervous system embryonal tumor, cerebellar astrocytoma, cerebral astrocytoma, cervical cancer, cholangiocarcinoma, chondroma, chondrosarcoma, chordoma, choriocarcinoma, choroid plexus papilloma, chronic lymphocytic leukemia, chronic monocytic leukemia, chronic myelogenous leukemia, chronic myeloproliferative disorder, chronic neutrophilic leukemia, clear cell renal cell carcinoma, clear-cell tumor, colon cancer, colorectal cancer, craniopharyngioma, cutaneous T-cell lymphoma, dermatofibrosarcoma protuberans, dermoid cyst, desmoplastic small round cell tumor, diffuse large B cell lymphoma, dysembryoplastic neuroepithelial tumor, embryonal carcinoma, endodermal sinus tumor, endometrial cancer, endometrial uterine cancer, endometrioid tumor, enteropathy-associated T-cell lymphoma, ependymoblastoma, ependymoma, epithelioid sarcoma, erythroleukemia, esophageal cancer, esthesioneuroblastoma, Ewing's sarcoma, extracranial germ cell tumor, extragonadal germ cell tumor, extrahepatic bile duct cancer, extramammary Paget's disease, fallopian tube cancer, fibroma, fibrosarcoma, follicular lymphoma, follicular thyroid cancer, gallbladder cancer, ganglioglioma, ganglioneuroma, gastric cancer, gastric lymphoma, gastrointestinal cancer, gastrointestinal carcinoid tumor, gastrointestinal stromal tumor, germ cell tumor, germinoma, gestational choriocarcinoma, gestational trophoblastic tumor, giant cell tumor of bone, glioblastoma multiforme, glioma, gliomatosis cerebri, glomus tumor, glucagonoma, gonadoblastoma, granulosa cell tumor, hairy cell leukemia, head and neck cancer, heart cancer, hemangioblastoma, hemangiopericytoma, hemangiosarcoma, hematological malignancy, hepatocellular carcinoma, hepatosplenic T-cell lymphoma, Hodgkin lymphoma, hypopharyngeal cancer, hypothalamic glioma, inflammatory breast cancer, intraocular melanoma, islet cell carcinoma, juvenile myelomonocytic leukemia, Kaposi's sarcoma, kidney cancer, klatskin tumor, krukenberg tumor, laryngeal cancer, lentigo maligna melanoma, leukemia, lip and oral cavity cancer, liposarcoma, lung cancer, luteoma, lymphangioma, lymphangiosarcoma, lymphoepithelioma, lymphoid leukemia, lymphoma, macroglobulinemia, malignant fibrous histiocytoma, malignant glioma, malignant mesothelioma, malignant peripheral nerve sheath tumor, malignant rhabdoid tumor, malignant triton tumor, malt lymphoma, mantle cell lymphoma, mast cell leukemia, mediastinal germ cell tumor, mediastinal tumor, medullary thyroid cancer, medulloblastoma, medulloepithelioma, melanoma, meningioma, merkel cell carcinoma, mesothelioma, metastatic squamous neck cancer with occult primary, metastatic urothelial carcinoma, mixed mullerian tumor, monocytic leukemia, mouth cancer, mucinous tumor, multiple endocrine neoplasia syndrome, multiple myeloma, mycosis fungoides, myelodysplastic disease, myeloid leukemia, myeloid sarcoma, myeloproliferative disease, myxoma, nasal cavity cancer, nasopharyngeal cancer, neoplasm, neurinoma, neuroblastoma, neurofibroma, neuroma, nodular melanoma, non-Hodgkin lymphoma, nonmelanoma skin cancer, non-small cell lung cancer, ocular oncology, oligoastrocytoma, oligodendroglioma, oncocytoma, optic nerve sheath meningioma, oral cancer, oropharyngeal cancer, osteosarcoma, ovarian cancer, ovarian epithelial cancer, ovarian germ cell tumor, ovarian low malignant potential tumor, pancoast tumor, pancreatic cancer, papillary thyroid cancer, papillomatosis, paraganglioma, paranasal sinus cancer, parathyroid cancer, penile cancer, perivascular epithelioid cell tumor, pharyngeal cancer, pheochromocytoma, pineal parenchymal tumor of intermediate differentiation, pineoblastoma, pituicytoma, pituitary adenoma, pituitary tumor, plasma cell neoplasm, pleuropulmonary blastoma, polyembryoma, precursor T-lymphoblastic lymphoma, primitive neuroectodermal tumor, prostate cancer, pseudomyxoma peritonei, rectal cancer, renal cell carcinoma, retinoblastoma, rhabdomyoma, rhabdomyosarcoma, Richter's transformation, sacrococcygeal teratoma, salivary gland cancer, sarcoma, schwannomatosis, sebaceous gland carcinoma, secondary neoplasm, seminoma, serous tumor, Sertoli-Leydig cell tumor, sex cord-stromal tumor, sezary syndrome, signet ring cell carcinoma, skin cancer, small blue round cell tumor, small cell carcinoma, small cell lung cancer, small cell lymphoma, small intestine cancer, soft tissue sarcoma, somatostatinoma, soot wart, spinal tumor, splenic marginal zone lymphoma, squamous cell carcinoma, stomach cancer, superficial spreading melanoma, supratentorial primitive neuroectodermal tumor, surface epithelial-stromal tumor, synovial sarcoma, T-cell acute lymphoblastic leukemia. T-cell large granular lymphocyte leukemia, T-cell leukemia, T-cell lymphoma, T-cell prolymphocytic leukemia, teratoma, terminal lymphatic cancer, testicular cancer, thecoma, throat cancer, thymic carcinoma, thymoma, thyroid cancer, transitional cell cancer of renal pelvis and ureter, transitional cell carcinoma, urachal cancer, urethral cancer, urogenital neoplasm, uterine sarcoma, uveal melanoma, vaginal cancer, verner morrison syndrome, verrucous carcinoma, visual pathway glioma, vulvar cancer, Waldenstrom's macroglobulinemia, Warthin's tumor, Wilms' tumor or any combination thereof.

In some embodiments, the methods of administering Formula I polymorph described herein are applied to the treatment of cancers of the adrenal glands, blood, bone marrow, brain, breast, cervix, colon, head and neck, kidney, liver, lung, ovary, pancreas, plasma cells, rectum, retina, skin, spine, throat or any combination thereof.

Certain embodiments contemplate a human subject such as a subject that has been diagnosed as having or being at risk for developing or acquiring a proliferative disorder condition. Certain other embodiments contemplate a non-human subject, for example a non-human primate such as a macaque, chimpanzee, gorilla, vervet, orangutan, baboon or other non-human primate, including such non-human subjects that can be known to the art as preclinical models. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present invention, a transgenic animal is utilized. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

### Example 1A: Preparation of 2-hydroxy-5-(methylthio)benzaldehyde (2)

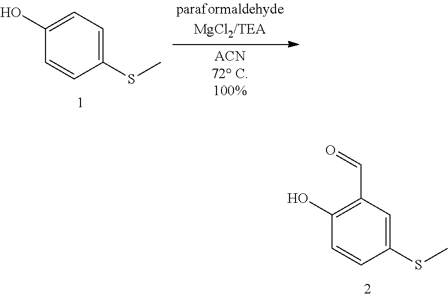

To a clean and dry 1000 L stainless steel reactor was charged acetonitrile (200 L) followed by 4-(methylthio)phenol 1 (20 kg, 142.85 mol) and the resulting mixture was stirred for 10-15 min. at room temperature to dissolve. To this was added paraformaldehyde (29.86 kg, 985.5 mol) at room temperature, followed by the portion-wise addition of magnesium chloride (21.71 kg, 228.57 mol) over 1 h. To this was added triethylamine (54.82 kg, 542.83 mol) and the reaction mixture was heated to 70° C. and maintained at this temperature for 3 h. After 3 h, the reaction was deemed complete by TLC analysis. The reaction mixture was cooled to 0-5° C. and the pH was adjusted to pH 3 using 3N aq. HCl. The aqueous layer was extracted with MTBE (4×100 L). The combined organic layer was washed with water (3×100 L), dried over sodium sulfate, filtered and distilled under vacuum to furnish 24.0 kg of 2-hydroxy-5-(methylthio)benzaldehyde (2) (100%, 86.1% HPLC purity). The product was characterized by HPLC, ¹H NMR and LCMS.

### Example 1B: Preparation of 6-(methylthio)-2-oxo-2H-chromene-3-carboxylic acid (3)

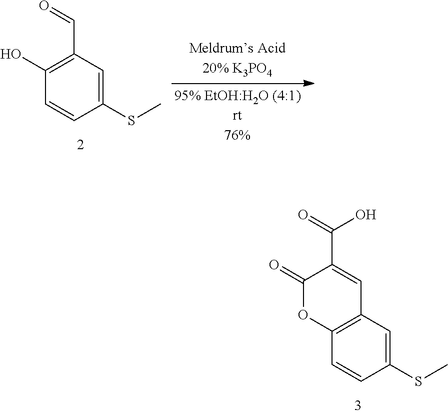 [SMILES: CSc1ccc(O)c(C=O)c1 ; CSc1ccc2oc(=O)c(C(=O)O)cc2c1]

To a clean 650 L reactor was added a solution of 2-hydroxy-5-(methylthio)benzaldehyde (2) (35.5 kg, 211.3 mol, combined batches) in ethanol (50 L) was charged Meldrum's acid (25.8 kg, 179.1 mol). To this was charged a solution of potassium phosphate (4.47 kg, 21.08 mol) in water (198 L) at room temperature The reaction mixture was stirred for 5-6 h at room temperature at which point the PH was adjusted to pH 3 using IN HCl. The solids obtained were filtered and washed with water (90 L) and acetone (40 L). The product was dried at room temperature to give 38 kg of 6-(methylthio)-2-oxo-2H-chromene-3-carboxylic acid (3) (76% yield, 97.7% purity by HPLC).

### Example 1C: Preparation of 3-(2-hydroxy-5-(methylthio)phenyl)propanoic acid (4)

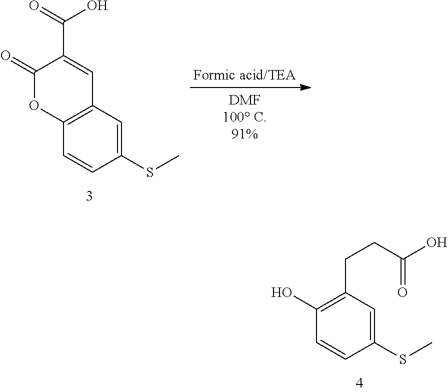 [SMILES: CSc1ccc2oc(=O)c(C(=O)O)cc2c1 ; CSc1ccc(O)c(CCC(=O)O)c1]

To a clean 650 L glass-lined reactor was charged a formic acid (44.44 kg, 966 mol) and DMF (162 L). To this solution was added triethylamine (39.01 kg, 386.4 mol) maintaining a temperature below 10° C. When the addition was complete, the mixture was heated to 100-105° C. To the reaction mixture was added 6-(methylthio)-2-oxo-2H-chromene-3-carboxylic acid (3) (38 kg, 161.01 mol) portion-wise. After the complete addition, the mixture was stirred at 100° C. for 2 h. The progress of the reaction was monitored by TLC. After the reaction was deemed complete by TLC, the reaction mixture was cooled to room temperature, then 6N NaOH solution (200 L) was added maintaining the temperature below 10° C. The reaction mixture was stirred at room temperature for 1 h, then washed with MTBE (2×150 L). The product-containing aqueous layer was adjusted to pH 4 using concentrated HCl (100 L). The aqueous layer was extracted with MTBE (3×150 L) and the combined organic layers were washed with brine (100 L). The organic layer was dried over sodium sulfate (15 kg), filtered and concentrated under reduced pressure. The solids obtained were triturated with toluene (100 L) at room temperature, and collected by filtration to give 31.16 kg of 3-(2-hydroxy-5-(methylthio)phenyl)propanoic acid (4) (91% yield, 94.5% HPLC purity).

### Example 1D: Preparation of 3-(2-(3-cyano-5-fluorophenoxy)-5-(methylthio)phenyl)propanoic acid 5

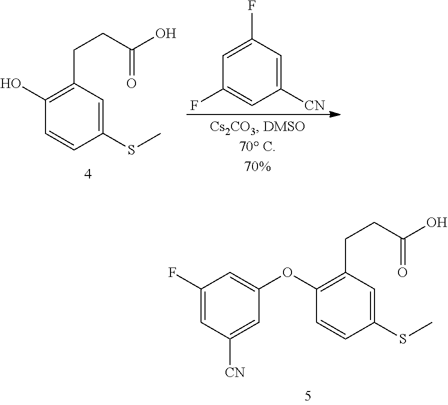

To a suspension of 3-(2-hydroxy-5-(methylthio)phenyl)propanoic acid (4) (27 kg, 127.3 mol) in DMSO (162 L) was added 3,5-difluorobenzonitrile (28.5 kg, 203.7 mol), followed by cesium carbonate (91 kg, 280 mol). The reaction mixture was heated to 75° C. and maintained at this temperature for 5 h. The reaction mixture was cooled to room temperature and water (150 L) was added. The mixture was washed with MTBE (3×100 L). The product-containing aqueous layer was adjusted to pH 3.5 using conc. HCl (60 L) maintaining the temperature below 10° C. The solids obtained were collected by filtration, washed with water. The solids were then washed with toluene (90 L) to give 29.3 kg of 3-(2-(3-cyano-5-fluorophenoxy)-5-(methylthio)phenyl)propanoic acid (5) (70% yield, 95% HPLC purity).

### Example 1E: Preparation of 3-fluoro-5-((7-(methylthio)-1-oxo-2,3-dihydro-1H-inden-4-yl)oxy)benzonitrile (6)

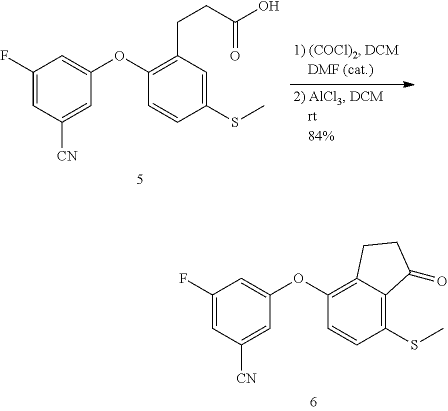 [SMILES: CSc1ccc(Oc2cc(F)cc(C#N)c2)c(CCC(=O)O)c1 ; CSc1ccc(Oc2cc(F)cc(C#N)c2)c2c1C(=O)CC2]

Note: A single batch of acid chloride was prepared, however to mitigate risk, the Friedel-Crafts acylation was performed in two batches. Following work-up, the two batches were combined and crystallized from ACN/H₂O to provide a single lot of 3-fluoro-5-((7-(methylthio)-1-oxo-2,3-dihydro-1H-inden-4-yl)oxy)benzonitrile (6).

Dichloromethane (42.6 kg) was charged to a glass-lined reactor, followed by 3-(2-(3-cyano-5-fluorophenoxy)-5-(methylthio)phenyl)propanoic acid (5) (24.0 kg, 72.4 mol) and DMF (0.03 kg). Oxalyl chloride (10.1 kg, 79.6 mol) was added to the resulting suspension over 2 hours (via a peristaltic pump) maintaining the temperature at 18-23° C. The resulting mixture was stirred at ambient temperature for 3 hours, at which point in-process analysis showed complete formation of the acid chloride (in-process sample quenched with methanol). The reaction mixture was concentrated by distillation at reduced pressure to a minimum stir volume. Dichloromethane (44.0 kg) was added and the resulting solution was drained into carboys, recording the weight of the acid chloride solution (net wt.: 72.8 kg). The reactor was rinsed with dichloromethane, which was discarded as chemical waste.

To the rinsed glass-lined reactor was added dichloromethane (103 kg). The reactor was purged with nitrogen and then aluminum chloride (9.65 kg, 72.5 mol) was added to the reactor. Approximately half of the acid chloride solution, prepared above, was added to the AlCl₃ suspension, via metered addition using a peristaltic pump, over 45 minutes, keeping the temperature below 25° C. (the addition is mildly exothermic). The reaction mixture was stirred for 30 min. at 18-22° C. at which point in-process analysis determined the conversion to product was complete (≧99.0%). The reaction mixture was cooled to −5-0° C., purified water was added over three hours using a peristaltic pump, maintaining the internal temperature below 2° C. (the quench is initially highly exothermic). Following the addition, the reaction mixture was stirred overnight at −5-0° C. The reaction mixture was warmed to 18-22° C., the stirring was stopped and the phases were allowed to separate. The lower organic phase was drained to labeled carboys. The aqueous phase was washed with DCM (60 kg), followed by two additional DCM washes (30 kg each), collecting each DCM wash into labeled carboys. A second reaction was repeated using the remaining acid chloride solution.

The combined organic phases were concentrated under reduced pressure until the DCM was removed (the product precipitates during the distillation). Acetonitrile (34.4 kg) was added and then an additional 18.8 kg of solvent was removed to ensure complete removal of the DCM. Additional acetonitrile (68.4 kg to total 84.0 kg) was added to the mixture. The resulting mixture was stirred for at least 12 hour at a jacket temperature of 35° C. Purified water (53.6 kg) was added over 15 minutes. The resulting mixture was stirred for 15-45 minutes and then approximately half of the product was collected by centrifugation. The filter cake was washed with a prepared wash solution consisting of acetonitrile (8.40 kg) and water (5.40 kg) and the resulting product filter cake was transferred to PE bags. The remaining suspension was transferred to the centrifuge and the product cake was washed with a prepared wash solution consisting of acetonitrile (8.40 kg) and water (5.40 kg) and the resulting product filter cake was transferred to PE bags. The product was dried at 35-40° C. in an air-vented drying oven until in-process analysis determined the LOD<0.5%. 19.17 kg (84%) of 3-fluoro-5-((7-(methylthio)-1-oxo-2,3-dihydro-1H-inden-4-yl)oxy)benzonitrile (6) was isolated as a beige solid material.

### Example 1F: Preparation of 3-fluoro-5-((7-(methylsulfonyl)-1-oxo-2,3-dihydro-1H-inden-4-yl)oxy)benzonitrile (7)

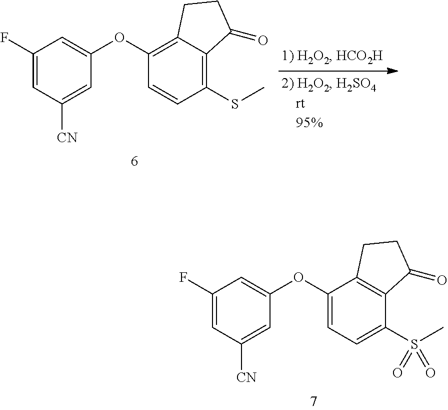 [SMILES: CSc1ccc(Oc2cc(F)cc(C#N)c2)c2c1C(=O)CC2 ; CS(=O)(=O)c1ccc(Oc2cc(F)cc(C#N)c2)c2c1C(=O)CC2]

To a suspension of 3-fluoro-5-((7-(methylthio)-1-oxo-2,3-dihydro-1H-inden-4-yl)oxy)benzonitrile (6) (18.20 kg, 1.0 eq.) in formic acid (94.5 kg), was added hydrogen peroxide solution (50% wt.) (4.30 kg, 1.1 eq.) maintaining the temperature below 32° C. The addition is exothermic. The reaction mixture was stirred at 28-32° C. for 50-70 minutes, until in-process analysis (HPLC) confirms less than 5.0 area % of 3-fluoro-5-((7-(methylthio)-1-oxo-2,3-dihydro-1H-inden-4-yl)oxy)benzonitrile (6) remaining.

Sulfuric acid (10.50 kg) and hydrogen peroxide solution (50% wt.) (4.30 kg, 1.1 eq.) was added below 32° C. The addition is exothermic. The reaction mixture was stirred at 28-32° C. for 2-3 hours. The mixture was seeded with crystals of 3-fluoro-5-((7-(methylsulfonyl)-1-oxo-2,3-dihydro-1H-inden-4-yl)oxy)benzonitrile (7) and stirring at 28-32° C. was continued for 12-24 hours, until in-process analysis (HPLC) confirmed conversion of ≧98.0% of 3-fluoro-5-((7-(methylthio)-1-oxo-2,3-dihydro-1H-inden-4-yl)oxy)benzonitrile (6) and the sulfoxide intermediate to 3-fluoro-5-((7-(methylsulfonyl)-1-oxo-2,3-dihydro-1H-inden-4-yl)oxy)benzonitrile (7).

Purified water (77.5 kg) was added while keeping the temperature below 32° C. The addition is exothermic. The suspension was cooled to 2-5° C. over 50-70 minutes and stirred for 15-60 minutes. The product was isolated on a centrifuge, washed with precooled purified water (139 kg), and dried in an air-vented drying cupboard at 40-45° C. to give 18.7 kg (93%) of 3-fluoro-5-((7-(methylsulfonyl)-1-oxo-2,3-dihydro-1H-inden-4-yl)oxy)benzonitrile (7) as a beige solid material.

### Example 1G: Preparation of 3-((2,2-difluoro-7-(methylsulfonyl)-1-oxo-2,3-dihydro-1H-inden-4-yl)oxy)-5-fluorobenzonitrile (9)

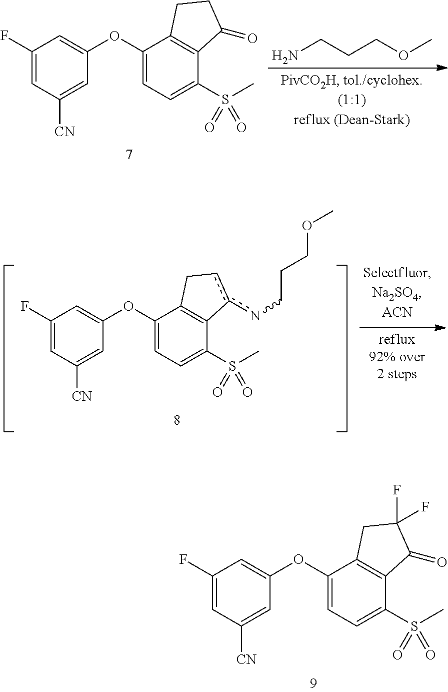

A mixture of 3-fluoro-5-((7-(methylsulfonyl)-1-oxo-2,3-dihydro-1H-inden-4-yl)oxy)benzonitrile (7) (9.00 kg, 1.0 eq.), 3-methoxypropylamine (3.05 kg, 1.3 eq.), pivalic acid (275 g, 0.1 eq.) in cyclohexane (51.5 kg) and toluene (57.0 kg) is heated to reflux and water is removed using a Dean-Stark condenser until at least the theoretical amount (0.47 kg) has been collected. The mixture is concentrated by distillation until approx. 114 L of solvents have been removed. Acetonitrile (14.3 kg) is added and the distillation is continued until additional 18 L of solvents have been removed. The “Imine” residue is cooled to 20-25° C.

A mixture of sodium sulfate (3.8 kg, 1.0 eq.) and Selectfluor® (23.15 kg, 2.5 eq.) in acetonitrile (48.8 kg) is heated to 68-70° C. and stirred for 5-20 minutes. The “Imine” solution 8 is added during 1-2 hours while keeping the temperature at 65-75° C. The mixture is stirred at 68-72° C. for 1-2 hours until minimum 99.0% (HPLC) conversion of 3-fluoro-5-((7-(methylsulfonyl)-1-oxo-2,3-dihydro-1H-inden-4-yl)oxy)benzonitrile to 3-((2,2-difluoro-7-(methylsulfonyl)-1-oxo-2,3-dihydro-1H-inden-4-yl)oxy)-5-fluorobenzonitrile (9) is achieved. The mixture is cooled to 20-25° C. and quenched with purified water (41.3 kg) and hydrochloric acid (37%) (8.65 kg, 3.4 eq.) at 15-25° C. The mixture is stirred at 20-25° C. for 30-90 minutes. The mixture is concentrated by distillation at reduced pressure until approx. 65 L has been removed. The residue is cooled to 19-21° C. The product is isolated on a centrifuge, washed with purified water (75.0 kg) and dried in an air-vented drying cupboard at 38-42° C. to give 8.95 kg (90%) of 3-((2,2-difluoro-7-(methylsulfonyl)-1-oxo-2,3-dihydro-1H-inden-4-yl)oxy)-5-fluorobenzonitrile (9) as a red-brown solid material.

### Example 1H: Preparation of (S)-3-((2,2-difluoro-1-hydroxy-7-(methylsulfonyl)-2,3-dihydro-1H-inden-4-yl)oxy)-5-fluorobenzonitrile (10)

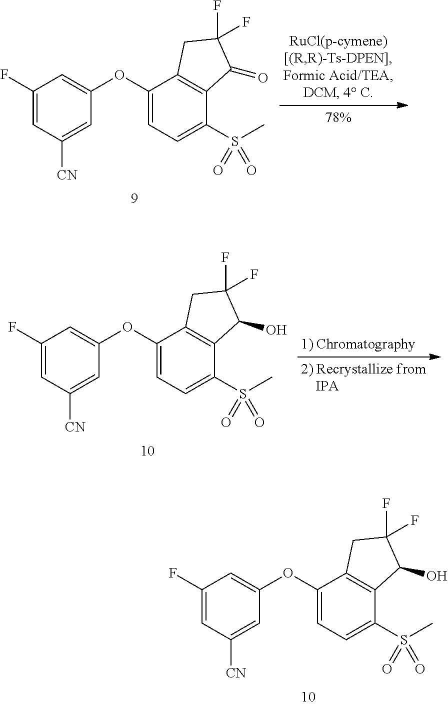 [SMILES: CS(=O)(=O)c1ccc(Oc2cc(F)cc(C#N)c2)c2c1C(=O)C(F)(F)C2 ; CS(=O)(=O)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)C(F)(F)C2]

a. Preparation of Form-A

Dichloromethane (76.5 kg), triethylamine (4.3 kg, 2.0 eq.) and formic acid (2.9 kg, 3.0 eq.) are mixed together at 0-10° C. under a nitrogen atmosphere. The temperature is adjusted to 0-5° C. and 3-((2,2-difluoro-7-(methylsulfonyl)-1-oxo-2,3-dihydro-1H-inden-4-yl)oxy)-5-fluorobenzonitrile (9) (8.00 kg, 1.0 eq.) and the Ruthenium catalyst (133 g, 0.01 eq.) are charged. The mixture is stirred at 3-5° C. for at least 12 hours until minimum 99.0% (HPLC) conversion of 3-((2,2-difluoro-7-(methylsulfonyl)-1-oxo-2,3-dihydro-1H-inden-4-yl)oxy)-5-fluorobenzonitrile (9) to (S)-3-((2,2-difluoro-1-hydroxy-7-(methylsulfonyl)-2,3-dihydro-1H-inden-4-yl)oxy)-5-fluorobenzonitrile (10) is achieved.

The mixture is washed with a mixture of purified water (8.0 kg) and sodium hydrogen carbonate (0.50 kg). The organic phase is stirred with sodium sulfate (3.5 kg) for at least 5 minutes and then also with activated carbon (0.80 kg) for at least 15 minutes.

A column is prepared from silica gel (39.2 kg) suspended in dichloromethane (90 kg) in a stainless steel pressure filter. The product suspension is loaded onto the column and eluted with a mixture of ethyl acetate (181 kg) and heptanes (90 kg). The fractions containing sufficiently pure product (determined by TLC) are selected for further processing.

The reaction and chromatography described above is repeated once more at the same scale.

The selected fractions from both chromatographies are combined and concentrated by distillation at reduced pressure until the solvent is removed and the residue starts to foam. 2-Propanol (25.6 kg) is charged, the residue is heated to 70-85° C. and stirred until all solids have dissolved. The mixture is slowly cooled with simultaneous seeding with (S)-3-((2,2-difluoro-1-hydroxy-7-(methylsulfonyl)-2,3-dihydro-1H-inden-4-yl)oxy)-5-fluorobenzonitrile (10) (Form A) seed crystals (1 g). The mixture is then further cooled to 5-10° C. and stirred for at least 6 hours. The product is isolated on a centrifuge, washed with precooled 2-propanol (18.8 kg) and dried in an air-vented drying cupboard at 38-42° C. to give 11.93 kg (70%) of (S)-3-((2,2-difluoro-1-hydroxy-7-(methylsulfonyl)-2,3-dihydro-1H-inden-4-yl)oxy)-5-fluorobenzonitrile (10) (Form A) as an off-white solid material.

b. Preparation of Form B

A solution of (S)-3-((2,2-difluoro-1-hydroxy-7-(methylsulfonyl)-2,3-dihydro-1H-inden-4-yl)oxy)-5-fluorobenzonitrile (10) (Form A, 3 kg) in dichloromethane (15.0 kg) is prepared. A column is prepared from silica gel (36.0 kg) suspended in dichloromethane (100 kg) in a stainless steel pressure filter. The product suspension is loaded onto the column and eluted with a mixture of dichloromethane (572.4 kg) and methyl tert-butyl ether (13.3 kg). The fractions containing sufficiently pure product (determined by TLC) are selected for further processing.

The selected fractions from both chromatographies are combined and concentrated by distillation at reduced pressure. 2-Propanol (14.2 kg) is charged and distillation is continued until 9 L more has been collected. The residue is heated to 70-85° C. and stirred until all solids have dissolved. The temperature is adjusted to 70-72° C. and the solution is seeded with (S)-3-((2,2-difluoro-1-hydroxy-7-(methylsulfonyl)-2,3-dihydro-1H-inden-4-yl)oxy)-5-fluorobenzonitrile (10) form B seed crystals (15 g). The mixture is stirred at 69-71° C. for at least 90 minutes and is then cooled to 18-22° C. over at least 2.5 hours. The suspension is stirred at 18-22° C. for at least 6 hours. The product is isolated on a sintered glass filter, washed with 2-propanol (4.7 kg) and dried in an air-vented drying cupboard at 38-42° C. to give 2.45 kg (81.5%) of (S)-3-((2,2-difluoro-1-hydroxy-7-(methylsulfonyl)-2,3-dihydro-1H-inden-4-yl)oxy)-5-fluorobenzonitrile (10) (Form B) as an off-white solid material.

c. Preparation of Form B

Dichloromethane (74.6 kg), triethylamine (4.2 kg, 2.0 eq.) and formic acid (2.8 kg, 3.0 eq.) are mixed together at 0-10° C. under a nitrogen atmosphere. The temperature is adjusted to 0-5° C. and 3-((2,2-difluoro-7-(methylsulfonyl)-1-oxo-2,3-dihydro-1H-inden-4-yl)oxy)-5-fluorobenzonitrile (9)(7.80 kg, 1.0 eq.) and the Ruthenium catalyst (130 g, 0.01 eq.) are charged. The mixture is stirred at 3-5° C. for at least 12 hours until minimum 99.0% (HPLC) conversion of 3-((2,2-difluoro-7-(methylsulfonyl)-1-oxo-2,3-dihydro-1H-inden-4-yl)oxy)-5-fluorobenzonitrile (9) to (S)-3-((2,2-difluoro-1-hydroxy-7-(methylsulfonyl)-2,3-dihydro-1H-inden-4-yl)oxy)-5-fluorobenzonitrile (10) is achieved.

The mixture is washed with:

1) A mixture of purified water (18.1 kg) and hydrochloric acid 37% (1.60 kg).
2) A mixture of purified water (18.15 kg) and sodium hydrogen carbonate (1.20 kg).
The organic phase is stirred with sodium sulfate (4.1 kg) for at least 5 minutes and then also with activated carbon (0.80 kg) for at least 15 minutes.

A column is prepared from silica gel (70.2 kg) suspended in dichloromethane (200 kg) in a 180 L stainless steel pressure filter. The product suspension is loaded onto the column and eluted with a mixture of tert-butyl methyl ether (24.5 kg) in dichloromethane (1056.5 kg). The fractions containing sufficiently pure 10 (TLC) are selected for further processing.

The reaction and chromatography described above is repeated once more at the same scale.

The selected fractions from both chromatographies are combined and concentrated by distillation at reduced pressure. 2-Propanol (43.1 kg) is charged and distillation is continued until 20 L more has been collected. The residue is heated to 70-85° C. and stirred until all solids have dissolved. The temperature is adjusted to 70-72° C. and the solution is seeded with (S)-3-((2,2-difluoro-1-hydroxy-7-(methylsulfonyl)-2,3-dihydro-1H-inden-4-yl)oxy)-5-fluorobenzonitrile (10) form B seed crystals (10-20 g). The mixture is stirred at 69-71° C. for at least 90 minutes and is then cooled to 5-10° C. over at least 4 hours. The suspension is stirred at 5-10° C. for at least 6 hours. The product is isolated on a centrifuge, washed with precooled 2-propanol (18.3 kg) and dried in an air-vented drying cupboard at 38-42° C. to give 12.3 kg (78.7%) of (S)-3-((2,2-difluoro-1-hydroxy-7-(methylsulfonyl)-2,3-dihydro-1H-inden-4-yl)oxy)-5-fluorobenzonitrile (10) (Form B) as an off-white solid material.

d. Additional procedures for preparation of Form A, Form B and Mixture

Form A Synthesis:

Formic acid (68.4 mL, 1.81 mol) was added to dichloromethane (2100 mL) at 0° C., followed by triethylamine (168.6 mL, 1.21 mol). 3-(2,2-difluoro-7-methylsulfonyl-1-oxo-indan-4-yl)oxy-5-fluoro-benzonitrile (230.0 g, 0.60 mol) was added and followed by RuCl(p-cymene)[(R,R)-Ts-DPEN] (1.9 g, 3.02 mmol) at 0° C. The reaction mixture was stirred at 3-5° C. for 10 hours, put in 4° C. refrigerator for 12 hours, then warmed to ambient temperature and stirred at ambient temperature for 2 hours. Saturated sodium bicarbonate (500 mL) was added. The organic layer was separated, dried (sodium sulfate), filtered and concentrated under reduced pressure. The residue obtained was purified by flash chromatography on silica gel 1:1 hexane/ethyl acetate to give 3-[(1S)-2,2-difluoro-1-hydroxy-7-methylsulfonyl-indan-4-yl]oxy-5-fluoro-benzonitrile (209 g, 91%) as solid (97% pure by HPLC and 96.5% ee by chiral HPLC).

3-[(1 S)-2,2-difluoro-1-hydroxy-7-methyl sulfonyl-indan-4-yl]oxy-5-fluoro-benzonitrile (209 g, 545.2 mmol) in 2-propanol (430 mL) was stirred at reflux for 10 minutes. All solids went into solution. The mixture was slowly cooled to ambient temperature with stirring and stirred at ambient temperature for 2 hours. The solid was collected by filtration, washed with IPA (200 mL) and dried to give 3-[(1S)-2,2-difluoro-1-hydroxy-7-methylsulfonyl-indan-4-yl]oxy-5-fluoro-benzonitrile (195.1 g, 93%) as white solid (99.3% pure by HPLC, 98.8% ee by chiral HPLC). LCMS ESI (−) 428 (M+HCO₂⁻). ¹HNMR (400 MHz, CDCl₃): δ 7.93 (d, 1H), 7.27-7.24 (m, 1H), 7.15-7.14 (m, 1H), 7.07-7.03 (m, 1H), 7.00 (d, 1H), 5.63-5.58 (m, 1H), 3.56-3.35 (m, 3H), 3.24 (s, 3H).

Form B Synthesis:

Formic acid (31.9 mL, 0.85 mol) was added to dichloromethane (1200 mL) at 0° C., followed by triethylamine (79.0 mL, 0.57 mol). 3-(2,2-difluoro-7-methylsulfonyl-1-oxo-indan-4-yl)oxy-5-fluoro-benzonitrile (108.0 g, 0.28 mol) was added and followed by RuCl(p-cymene)[(R,R)-Ts-DPEN] (0.90 g, 1.42 mmol) at 0° C. The reaction mixture was put in 4° C. refrigerator for 24 hours and warmed to ambient temperature and stirred at ambient temperature for 2 hours. Water (300 mL) was added. The organic layer was separated, dried (sodium sulfate), filtered and concentrated under reduced pressure. The residue obtained was purified by careful flash chromatography on silica gel 1:1 hexane/ethyl acetate to give 3-[(1S)-2,2-difluoro-1-hydroxy-7-methylsulfonyl-indan-4-yl]oxy-5-fluoro-benzonitrile (102.5 g, 94%) as solid (97.9% ee by chiral HPLC and >99.0% pure by HPLC).

3-[(1S)-2,2-difluoro-1-hydroxy-7-methylsulfonyl-indan-4-yl]oxy-5-fluoro-benzonitrile (102.0 g, 0.27 mol) in 2-Propanol (300 mL) was stirred at reflux for 10 minutes. All solids went into solution. The mixture was slowly cooled to ambient temperature with stirring and stirred at ambient temperature for 2 hours. The solid was collected by filtration, washed with IPA (200 mL) and dried to give 3-[(1S)-2,2-difluoro-1-hydroxy-7-methylsulfonyl-indan-4-yl]oxy-5-fluoro-benzonitrile (97.8 g, 95%) as white solid (99.5% ee by chiral HPLC, 99.7% pure by HPLC). Melting point: 138.0-138.5° C.

The mixture of Form A and B synthesis:

Formic acid (66.2 mL, 1.75 mol) was added to dichloromethane (1200 mL) at 0° C., followed by triethylamine (163.0 mL, 1.17 mmol). 3-(2,2-difluoro-7-methylsulfonyl-1-oxo-indan-4-yl)oxy-5-fluoro-benzonitrile (223.0 g, 0.58 mol) was added and followed by RuCl(p-cymene)[(R,R)-Ts-DPEN] (1.86 g, 2.92 mmol) at 0° C. The reaction mixture was stirred at 3-5° C. for 10 hours, put in 4° C. refrigerator for 12 hours and stirred at 3-5° C. for 3 hours and slowly warmed to ambient temperature (1 hour) and stirred at ambient temperature for 2 hours. Saturated sodium bicarbonate (500 mL) was added. The organic layer was separated, dried (sodium sulfate), filtered and concentrated under reduced pressure. The residue obtained was purified by flash chromatography on silica gel 1:1 hexane/ethyl acetate to give 3-[(1 S)-2,2-difluoro-1-hydroxy-7-methylsulfonyl-indan-4-yl]oxy-5-fluoro-benzonitrile (217.2 g, 97%) as solid (˜98% pure by HPLC, 97.7% ee by chiral HPLC).

### Example 2: XRPD Analysis

XRPD analysis was carried out on a Bruker AXS D8 Advance X-ray diffractometer, scanning the samples between 23 and 40° 2-theta. Material was gently compressed on a glass disc inserted into the sample holder. The sample was then loaded into a Bruker AXS D8 diffractometer running in reflection mode and analyzed, using the following experimental conditions.

The XRPDs of Forms A and B are shown in FIGS. 2 and 13 respectively.

### Example 3: Polarized Light Microscopy (PLM)

The presence of crystallinity (birefringence) was determined using an Olympus BX50 polarizing microscope, equipped with a Motic camera and image capture software (Motic Images Plus 2.0). All images were recorded using the 20× objective, unless otherwise stated.

### Example 4: Hot-Stage Microscopy (HSM)

The sample was placed in a THM Linkam hot-stage and heated at a rate of 10° C./min from room temperature (ca. 22° C.) to 110° C., then 5° C./min from 110° C. to 125° C. and 1° C./min from 125° C. to 146° C. Thermal events were monitored visually using an Olympus BX50 microscope, equipped with a Motic camera and image capture software (Motic Images Plus 2.0). All images were recorded using a 10× objective, unless otherwise stated.

### Example 5A: Thermogravimetric Analysis (TGA)

Approximately 5 mg of material was weighed into an open aluminum pan and loaded into a simultaneous thermogravimetric/differential thermal analyzer (TG/DTA) and held at room temperature. The sample was then heated at a rate of 10° C./min from 25° C. to 300° C. during which time the change in sample weight was recorded along with any differential thermal events (DTA). Nitrogen was used as the purge gas, at a flow rate of 100 cm³/min.

### Example 5B: Thermogravimetric Analysis—Residue on Ignition (TGA/ROI)

Approximately 5 mg of material was weighed into an open ceramic pan and loaded into a simultaneous thermogravimetric/differential thermal analyzer (TG/DTA) and held at room temperature. The sample was then heated at a rate of 40° C./min from 25° C. to 800° C. during which time the change in sample weight was recorded along with any differential thermal events (DTA). The temperature was then maintained at 800° C. for 20, 40 or 60 min. Nitrogen was used as the purge gas, at a flow rate of 100 cm³/min.

### Example 5C: Differential Scanning Calorimetry (DSC)

Approximately 5 mg of material was weighed into an aluminum DSC pan and sealed non-hermetically with a pierced aluminum lid. The sample pan was then loaded into a Seiko DSC6200 (equipped with a cooler) cooled and held at 25° C. Once a stable heat-flow response was obtained, the sample and reference were heated to 270° C. at scan rate of 10° C./min and the resulting heat flow response monitored.

### Example 6: Differential Scanning Calorimetry (DSC)—Cycling Method

Approximately, 5 mg of material was weighed into an aluminum DSC pan and sealed non-hermetically with a pierced aluminum lid. The sample pan was then loaded into a Seiko DSC6200 (equipped with a cooler) cooled and held at 25° C. Once a stable heat-flow response was obtained, the sample and reference were heated to 145° C. at scan rate of 10° C./min, then allowed to cool back to 25° C. The sample and reference were then reheated to 270° C. and the resulting heat flow response monitored.

### Example 7: Karl Fischer Coulometric Titration (KF)

ca. 10-15 mg of solid material was accurately weighed into a vial. The solid was then manually introduced into the titration cell of a Mettler Toledo C30 Compact Titrator. The vial was back-weighed after the addition of the solid and the weight of the added solid entered on the instrument. Titration was initiated once the sample had fully dissolved in the cell. The water content was calculated automatically by the instrument as a percentage and the data printed.

### Example 8: Infrared Spectroscopy (IR)

Infrared spectroscopy was carried out on a Bruker ALPHA P spectrometer. Sufficient material was placed onto the center of the plate of the spectrometer and the spectra were obtained using the following parameters:
Resolution: 4 cm⁻¹
Background Scan Time: 16 scans
Sample Scan Time: 16 scans
Data Collection: 4000 to 400 cm⁻¹
Result Spectrum: Transmittance
Software: OPUS version 6

### Example 9: ¹H Nuclear Magnetic Resonance (¹H NMR)

¹HNMR experiments were performed on a Bruker AV400 (frequency: 400 MHz). Experiments were performed in deuterated DMSO and each sample was prepared to ca. 10 mM concentration.

### Example 10: Dynamic Vapor Sorption (DVS)

Approximately 10 mg of sample was placed into a mesh vapor sorption balance pan and loaded into a DVS-1 dynamic vapor sorption balance by Surface Measurement Systems. The sample was subjected to a ramping profile from 0-90% relative humidity (RH) at 10% increments, maintaining the sample at each step until a stable weight had been achieved (99.5% step completion). After completion of the sorption cycle, the sample was dried using the same procedure, down to 0% RH. The weight change during the sorption/desorption cycles were plotted, allowing for the hygroscopic nature of the sample to be determined.

### Example 11: High Performance Liquid Chromatography-Ultraviolet Detection (HPLC-UV)

Initial Method:
Instrument: HPLC—Agilent 1100 with UV detector
Column: Phenomenex Luna C18, 5 μm, 4.6×150 mm
Column Temperature: 20° C.
Autosampler Temperature: 20° C.
UV wavelength: 234 nm
Injection Volume: 5 μL
Flow Rate: 1.0 mL/min

Mobile Phase A: Water containing 0.1% Formic Acid

Mobile Phase B: Acetonitrile containing 0.1% Formic Acid

Gradient Program:

| Time (minutes) | Solvent B [%] |
|---|---|
| 0 | 30 |
| 20.0 | 70 |
| 25.0 | 95 |
| 30.0 | 95 |
| 30.1 | 30 |
| 35.0 | 30 |

General Method:
Instrument: HPLC—Agilent 1100 with UV detector
Column: Agilent Zorbax Eclipse XDB-C18, 5 μm, 4.6×250 mm
Column Temperature: 40° C.
Autosampler Temperature: 20° C.
UV wavelength: 232 nm
Injection Volume: 10 μL
Flow Rate: 1.0 mL/min
Mobile Phase A: Water
Mobile Phase B: Acetonitrile

Gradient Program:

| Time (minutes) | Solvent B [%] |
|---|---|
| 0 | 30 |
| 30.0 | 45 |
| 50.0 | 90 |
| 52.0 | 90 |
| 52.1 | 30 |
| 60.0 | 30 |

### Example 12: Solubility Screen for Form a in Organic Solvents

A solubility screen for Form A was conducted in 24 solvents. About 5 mg of Form A was placed in each of 24 vials and 5 volume aliquots of the appropriate solvent systems listed in Table 1 were added to the appropriate vial. Between each addition, the mixture was checked for dissolution by visual inspection, and if no dissolution was apparent, the mixture was heated to ca. 40° C. and checked again. This procedure was continued until dissolution was observed or until 100 volumes of solvent had been added.

The results are shown in Table. Form A appeared to be highly soluble (above ˜100 mg/mL) in a number of solvents, Form A is insoluble in water, such that hydrophobic behavior is observed, diisopropyl ether, heptane and toluene. These solvents were identified as possible anti-solvents for the primary polymorph screen. Additional solvent/water mixtures suitable for freeze drying were tested. Sufficient solubility was observed in THF:Water (50%), but separation into an API-saturated THF layer and API-free aqueous layer was observed.

TABLE 1
Form A Solvent Solubility Results in 24 Solvents

| | Solvent | Solubility (mg/mL) |
|---|---|---|
| 1 | Acetone | >200 |
| 2 | Acetone: Water (5%) | >200 |
| 3 | Acetone: Water (20%) | >200 |
| 4 | Acetonitrile | >200* |
| 5 | Acetonitrile: Water (10%) | >200 |
| 6 | Dichloromethane | 80 |
| 7 | Diisopropyl ether | >2* |
| 8 | Dimethylformamide | >200 |
| 9 | Dimethylsulfoxide | 160 |
| 10 | 1,4-Dioxane | >200* |
| 11 | Ethanol | 60 |
| 12 | Ethyl acetate | 160 |
| 13 | Heptane | <2 |
| 14 | Isopropyl acetate | 90 |
| 15 | Methanol | 90 |
| 16 | Methyl acetate | >200 |
| 17 | Methylethyl ketone | >200 |
| 18 | Methyl isobutyl ketone | 160* |
| 19 | N-Methyl-2-pyrrolidone | 130 |
| 20 | 2-Propanol | 9 |
| 21 | tert-Butylmethyl ether | 35 |
| 22 | Tetrahydrofuran | >200 |
| 23 | Toluene | >2 |
| 24 | Water | <2 |
| 25 | Acetone: Water (60%) | <2 |
| 26 | Ethanol: Water (50%) | <2 |
| 27 | Methanol: Water (50%) | <2 |
| 28 | THF: Water (50%) | 100 |

*Solubility observed at 40° C.

### Example 13: 7-Day Stability Study

The stability of Forms A and B were measured following storage for 7-days at ambient, 40° C./75% RH and 80° C. About 15 mg of Forms A and B were individually placed in each of 3 vials and stored uncapped for 7 days under the following conditions; ambient, 40° C./75% RH, 80° C. Following the 7 days, XRPD analysis was performed on each sample to determine its form and crystallinity and HPLC analysis was conducted to determine the sample purity. XRPD analysis showed no signs of form change for either Form A or Form B from the three conditions tested. Form A appeared to lose crystallinity upon heating at 80° C. over 7 days, although the material did maintain a purity of 99.0% by HPLC over all three conditions. Form B maintained crystallinity over the three conditions, although a slight drop in purity (99.7% to 99.3%) was noted for the sample stored at 80° C.

### Example 14: Competitive Slurries

Competitive slurry experiments were performed in order to determine the thermodynamically most stable form. Slurry experiments were performed at ambient, 40° C. and 60° C. temperatures using 2-propanol, tert-butyl methyl ether, toluene and ethanol as solvents.

(i) Competitive Slurry at Ambient

Saturated solutions of 2-propanol, tert-butylmethyl ether, toluene and ethanol were prepared at ambient temperature. ca. 25 mg of Form A and Form B were weighed into a vial to create 50 mg 50:50 wt/wt mixture of the two batches. The appropriate solvent was added to the mixture and the resulting slurry was stirred for 48 h at ambient temperature, before the solid was analyzed by XRPD.

(ii) Competitive Slurry at 40° C.

Saturated solutions of 2-propanol, tert-butylmethyl ether, toluene and ethanol were prepared at 40° C. ca. 25 mg of Form A and Form B were weighed into a vial to create 50 mg 50:50 wt/wt mixture of the two batches. The appropriate solvent was added to the mixture and the resulting slurry was stirred for 48 h at 40° C., before the solid was analyzed by XRPD.

(iii) Competitive Slurry at 60° C.

Saturated solutions of 2-propanol, toluene and ethanol were prepared at 60° C. ca. 25 mg of Form A and Form B were weighed into a vial to create 50 mg 50:50 wt/wt mixture of the two batches. The appropriate solvent was added to the mixture and the resulting slurry was stirred for 48 h at 60° C., before the solid was analyzed by XRPD.

The competitive slurry results are summarized in Table 2. Form B was the dominant form in all solvents at all temperatures. Initial experiments run in ethanol and 2-propanol at 60° C. showed a mixture of forms with 2-propanol exhibiting primarily Form B with some Form A present and ethanol exhibiting primarily Form A with some Form B present. Upon retesting the experiment in ethanol, only Form B was observed. It was suspected the initial result was due to material sitting on the solvent interface in the reaction vial rather than in the bulk solvent. Since, all experiments resulted in conversion to Form B, it was concluded that Form B was the most thermodynamically form.

TABLE 2
Competitive Slurry Results

| | Temperature | | | |
|---|---|---|---|---|
| Solvent | Ambient | 40° C. | 60° C. | Repeat 60° C. |
| 2-Propanol | Form B | Form B | Mixture^{a} | NR |
| TBME | Form B | Form B | NR | NR |
| Toluene | Form B | Form B | Form B | NR |
| Ethanol | Form B | Form B | Mixture^{b} | Form B^{b} |

^{a}Primarily Form B
^{b}Initially, exhibited primarily Form A with some Form B present. It was suspected the initial result was due to material sitting on the solvent interface in the reaction vial rather than in the bulk solvent. Condition was repeated and only Form B was observed.
NR = Not Run

### Example 15: Aqueous Solubility

The aqueous solubility of several lots of Forms A and B were measured. Form A or B was weighed into a HPLC vial, before the addition of H₂O (500 μL) and the resulting mixture was placed on the belly-shaker for 24 h mixing. The sample was filtered using a syringe filter and the filtrate was analyzed by HPLC to determine the solubility. The remaining solid was analysis by XRPD to determine its form and crystallinity. Solubility data for micronized materials was also collected. As noted in Table 3, slight variations in aqueous solubility were noted, particularly for the micronized samples.

TABLE 3
Aqueous Solubility of the compound of Formula I

| | pH 1.2 | pH 6.5 | pH 7.4 | FaSSIF | FeSSIF |
|---|---|---|---|---|---|
| | (μg) | (μg) | (μg) | (μg) | (μg) |
| Form A | 29 | 25 | 26 | 63 | 86 |
| Form B | 25 | 24 | 24 | 42 | 61 |
| Form A, micronized | 37 | 35 | 35 | 68 | 97 |
| Form B, micronized | 21 | 20 | 22 | 42 | 62 |

FaSSIF = Fasted State Simulated Small Intestinal Fluid
FeSSIF = Fed State Simulated Small Intestinal Fluid

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising predominantly polymorph Form A of a compound of Formula I:

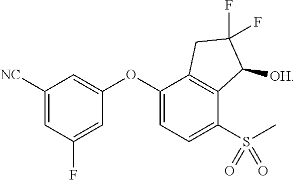 [SMILES: CS(=O)(=O)c1ccc(Oc2cc(F)cc(C#N)c2)c2c1[C@H](O)C(F)(F)C2]

2. The composition of claim 1, wherein greater than about 90% of the compound of Formula I is polymorph Form A.

3. The composition of claim 1, wherein greater than about 95% of the compound of Formula I is polymorph Form A.

4. The composition of claim 1, wherein greater than about 99% of the compound of Formula I is polymorph Form A.

5. The composition of any one of the preceding claims, wherein said polymorph Form A is characterized by having X-ray powder diffraction (XRPD) peaks at about 17.8, about 18.5, about 20.3 and about 21.2 degrees 2θ.

6. The composition of any one of the preceding claims, wherein said polymorph Form A is characterized by having X-ray powder diffraction (XRPD) peaks at about 6.8, about 15.9, about 17.8, about 18.5, about 20.3, about 20.5, about 21.2, about 22.1, about 22.7 and about 24.7 degrees 2θ.

7. The composition of any one of the preceding claims, wherein the polymorph Form A comprises cubic crystals.

8. The composition of any one of the preceding claims, wherein the polymorph Form A has a chemical purity of greater than about 90%.

9. The composition of any one of the preceding claims, wherein the polymorph Form A has a chemical purity of greater than about 95%.

10. The composition of any one of the preceding claims, wherein the polymorph Form A has a chemical purity of greater than about 99%.

11. The composition of any one of the preceding claims, wherein the chemical purity of the polymorph Form A is measured by HPLC analysis.

12. The composition of any one of the preceding claims, wherein the polymorph Form A has an enantiomeric purity of greater than about 90%.

13. The composition of any one of the preceding claims, wherein the polymorph Form A has an enantiomeric purity of greater than about 95%.

14. The composition of any one of the preceding claims, wherein the polymorph Form A has an enantiomeric purity of greater than about 99%.

15. The composition of any one of the preceding claims, wherein the polymorph Form A is dry.

16. The composition of any one of the preceding claims, wherein the polymorph Form A is non-solvated.

17. The composition of any one of the preceding claims, wherein the polymorph Form A is non-hydrated.

18. The composition of any one of the preceding claims, wherein the polymorph Form A is non-hygroscopic.

19. A composition comprising polymorph Form B of a compound of Formula I:

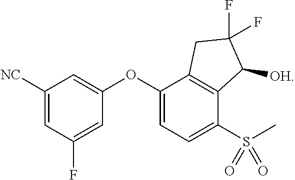 [SMILES: CS(=O)(=O)c1ccc(Oc2cc(F)cc(C#N)c2)c2c1[C@H](O)C(F)(F)C2]

20. The composition of claim 19, wherein said polymorph Form B is characterized by having X-ray powder diffraction (XRPD) peaks at about 24.3 degrees 2θ.

21. The composition of claim 19 or 20, wherein said polymorph Form B is characterized by having X-ray powder diffraction (XRPD) peaks at about 12.8, about 14.8, about 17.6 and about 24.3 degrees 2θ.

22. The composition of any one of claims 19-21, wherein said polymorph Form B is characterized by having X-ray powder diffraction (XRPD) peaks at about 12.8, about 14.8, about 17.6, about 20.1, about 20.9, about 22.2, about 24.3, about 25.0, about 25.6 and about 28.1 degrees 2θ.

23. The composition of any one of claims 19-22, wherein the polymorph Form B comprises thin rod or needle like crystals.

24. The composition of any one of claims 19-23, wherein the polymorph Form B has a chemical purity of greater than about 90%.

25. The composition of any one of claims 19-24, wherein the polymorph Form B has a chemical purity of greater than about 95%.

26. The composition of any one of claims 19-25, wherein the polymorph Form B has a chemical purity of greater than about 99%.

27. The composition of any one of claims 19-26, wherein the chemical purity of the polymorph Form B is measured by HPLC analysis.

28. The composition of any one of claims 19-27, wherein the polymorph Form B has an enantiomeric purity of greater than about 90%.

29. The composition of any one of claims 19-28, wherein the polymorph Form B has an enantiomeric purity of greater than about 95%.

30. The composition of any one of claims 19-29, wherein the polymorph Form B has an enantiomeric purity of greater than about 99%.

31. The composition of any one of claims 19-30, wherein the polymorph Form B is dry.

32. The composition of any one of claims 19-31, wherein the polymorph Form B is non-solvated.

33. The composition of any one of claims 19-32, wherein the polymorph Form B is non-hydrated.

34. The composition of any one of claims 19-33, wherein the polymorph Form B is non-hygroscopic.

35. The composition of any one of claims 19-34, wherein the composition further comprises polymorph Form A.

36. The composition of any one of claims 19-35, wherein the composition further comprises amorphous form of Formula I.

37. The composition of any one of claims 19-36, wherein the composition further comprises polymorph Form A and amorphous form of Formula I.

38. The composition of any one of claims 19-37, wherein the ratio of polymorph Form B to the total amount of non-B polymorphs is greater than about 1:1.

39. The composition of any one of claims 19-37, wherein the ratio of polymorph Form B to the total amount of non-B polymorphs is greater than about 9:1.

40. The composition of any one of claims 19-37, wherein the ratio of polymorph Form B to the total amount of non-B polymorphs is greater than about 99:1.

41. The composition of any one of claims 19-40, wherein said composition is at least 98% by weight compound of Formula I.

42. A pharmaceutical composition comprising a composition of any one of claims 1 to 41 and a pharmaceutically acceptable carrier.

43. A method of inhibiting HIF-2α activity in a cell, comprising contacting said cell with an effective amount of a composition or pharmaceutical composition of any one of claims 1-42.

44. A method of treating a neoplastic condition in a subject, comprising administering to said subject a therapeutically effective amount of a composition or pharmaceutical composition of any one of claims 1-42.

45. A method of treating renal cell carcinoma (RCC) in a subject, comprising administering to said subject a therapeutically effective amount of a composition or pharmaceutical composition of any one of claims 1-42.
